(19) United States
(12) Patent Application Publication
(10) Pub. No.: US 2022/0195002 A1
(43) Pub. Date: Jun. 23, 2022

(54) PROCESS FOR PREPARING GRANULOCYTE-COLONY STIMULATING FACTOR

(71) Applicant: Tanvex BioPharma USA, Inc., San Diego, CA (US)

(72) Inventor: Jennifer Renee Hopp, San Diego, CA (US)

(21) Appl. No.: 17/605,179

(22) PCT Filed: Apr. 20, 2020
(86) PCT No.: PCT/US20/28996

§ 371 Date: Oct. 20, 2021

Related U.S. Application Data
(60) Provisional application No. 62/838,226, filed on Apr. 24, 2019.

(51) Int. Cl. C07K 14/535

(52) U.S. Cl. CPC C07K 14/535

(57) ABSTRACT

Provided herein, inter alia, are compositions and methods for the isolation and/or purification of granulocyte colony-stimulating factor (G-CSF) from inclusion bodies (IBs). Some embodiments of the disclosure relate to a method for preparing biologically active and correctly folded G-CSF with improved purity and/or functional activity by optimizing the folding of recombinant G-CSF contained in the IBs. Also provided are G-CSF obtained by such methods, pharmaceutical compositions containing the same, as well as methods for the treatment and/or prevention of a disease in a subject in need thereof.

### CROSS-REFERENCE TO RELATED APPLICATIONS

This application claims the benefit of priority to U.S. Provisional Patent Application Ser. No. 62/838,226, filed on Apr. 24, 2019. The disclosure of the above-referenced application is herein expressly incorporated by reference it its entirety, including any drawings.

### FIELD

The present disclosure generally relates to a process for the isolation and/or preparation of granulocyte colony-stimulating factor (G-CSF), and particularly recombinant human G-CSF from inclusion bodies (IBs) produced in prokaryotic cells.

### BACKGROUND

Granulocyte-colony stimulating factor (G-CSF), also known as colony-stimulating factor 3 (CSF-3), belongs to the group of colony stimulating factors which regulate the differentiation and proliferation of hematopoietic precursor cells and activation of mature neutrophils. In particular, G-CSF is reported to stimulate the bone marrow to produce granulocytes and stem cells and release them into the bloodstream, and therefore is often used in medicine in the field of hematology and oncology.

With the advent of recombinant technology, techniques for the genetic transformation of various host organisms for the purposes of producing specific proteins through the expression of heterologous or foreign genes have been extensively developed. In particular, the synthesis of biologically active recombinant proteins is the key issue for the manufacture of commercially important polypeptides. Frequently, recombinant polypeptides are not able to fold properly within the host cell and form amorphous protein aggregates usually consisting of misfolded, often denatured polypeptides. These aggregates are known as inclusion bodies (IBs) or refractile bodies since they appear as highly refractile areas when the cells are observed microscopically. In the case of G-CSF, it can now be recombinantly produced in various eukaryotic organisms, e.g., yeasts and mammalian cells, and prokaryotic organisms such as bacteria. The form of recombinantly produced G-CSF depends on the type of host organism used for expression. When G-CSF is recombinantly produced in non-mammalian host cells, particularly prokaryotic cells, the G-CSF protein is generally expressed in a non-native form, which is often component of inactive IBs with limited solubility. Often, IBs formed in recombinant host cells have complex secondary structures and are densely aggregated. In the case of G-CSF, the production of biologically active recombinant G-CSF protein from inactive IBs expressed in commonly used non-mammalian host cells has also been reported to be challenging.

Generally, the purification of the recombinantly expressed proteins from IBs includes extraction of IBs from the host cells followed by the solubilization of the purified IBs. It is often difficult to recover the recombinant protein from IBs due to various technical problems associated with the initial harvesting, solubilization, and renaturation steps. Existing processes for the isolation and purification of recombinant G-CSF proteins produced in IBs generally are complex, lengthy and unit costs are high. In addition, these processes often incorporate strong denaturing agents, strong reducing agents, a redox reaction, and/or heavy metals. Furthermore, many of these agents have challenges including being costly at a large production scale and caustic in stainless steel manufacturing plants.

Therefore, to overcome the various technical issues associated with the manufacture of recombinant G-CSF, there is a need for improved production methods and scalable procedures that are cost-effective, stable for high recovery of G-CSF, and industrially applicable, e.g. can be implemented at a commercial production scale.

### SUMMARY

Provided herein, inter alia, are compositions and methods for the preparation and/or isolation of granulocyte colony-stimulating factor (G-CSF) in highly purified and active form. As described in greater detail below, the disclosed methods are particularly useful for G-CSF expressed in bacterial expression systems, and more particularly in bacterial systems in which G-CSF is expressed in the form of inclusion bodies within the bacterial cell. Also provided are G-CSF obtained by such methods, pharmaceutical compositions containing the same, as well as methods for the treatment and/or prevention of a disease in a subject in need thereof.

In one aspect, some embodiments of the disclosure relate to a method for isolating granulocyte colony-stimulating factor (G-CSF) from inclusion bodies (IBs), including: (a) solubilizing the G-CSF contained in the IBs with a solubilization buffer including a denaturing agent; and (b) initiating folding of the solubilized G-CSF by diluting, via a sequential stepwise dilution process, the solubilizate from (a) with a folding buffer including only the reduced form of a thiol redox pair to obtain folded G-CSF.

In one aspect, some embodiments of the disclosure relate to a method for preparing biologically active G-CSF, including: (a) solubilizing IBs containing G-CSF with a solubilization buffer including a denaturing agent; and (b) initiating folding of the solubilized G-CSF by diluting, via a sequential stepwise dilution process, the solubilizate from (a) with a folding buffer including only the reduced form of a thiol redox pair to obtain folded G-CSF with improved purity and/or functional activity.

Implementations of embodiments of the methods for isolating and/or preparing G-CSF according to the present disclosure can include one or more of the following features. In some embodiments, the obtained G-CSF includes biologically active, correctly folded G-CSF with a purity of greater than 80%. In some embodiments, the methods further including recovering the folded G-CSF. In some embodiments, the IBs containing G-CSF are suspended in a suspension buffer prior to solubilization. In some embodiments, the suspension buffer includes about 20 mM to 60 mM Tris at pH ranging from about 7.0 to 8.0. In some embodiments, the suspension buffer includes about 40 mM Tris at pH of about 7.6.

In some embodiments, the denaturing agent in the solubilization buffer includes a mild denaturing detergent, a strong denaturing detergent, an ionic detergent, or any combination thereof. In some embodiments, the denaturing agent in the solubilization buffer includes N-lauroyl sarcosine (sarkosyl), sodium dodecyl sulfate (SDS), sodium lauryl sulfate, polyoxyethylene poloxypropylene glycol, cocoamphoacetate, lithium dodecyl sulfate, sodium octyl sulfate, deoxycholic acid, sodium cholate hydrate, sodium deoxycholate, sodium glycocholate, sodium taurodeoxycholate, or any combination thereof. In some embodiments, the denaturing agent is an anionic detergent. In some embodiments, the anionic detergent in the solubilization buffer is sarkosyl. In some embodiments, sarkosyl is present in the solubilization buffer in an amount ranging from about 0.2% to about 5.0% by weight. In some embodiments, sarkosyl is present in the solubilization buffer in an amount of about 0.2%, about 0.56%, about 1.0%, or about 2.0% by weight. In some embodiments, the solubilization buffer includes about 20 mM to 60 mM Tris, about 0.2% to about 5% sarkosyl, at pH ranging from about 7.5 to about 9.0. In some embodiments, the solubilization buffer includes about 40 mM, about 2.0% sarkosyl, at pH of about 8.4. In some embodiments, the volume ratio of the suspension buffer to the solubilization buffer is adjusted such that the final pH is about 7.5 to about 7.8.

In some embodiments, the sequential stepwise dilution process includes gradually reducing the concentration of the denaturing agent in the solubilizate from (a). In some embodiments, the process of gradually reducing the denaturing agent concentration includes one or more of the following operations: (i) mixing the solubilization buffer with the suspension buffer in which the IBs containing G-CSF are suspended; (ii) diluting the solubilizate from (i) with water for injection (WFI) to form a diluted solubilizate; (iii) adding the folding buffer to the diluted solubilizate from (ii); and (iv) further diluting the folding mixture from (iii) with WFI.

In some embodiments, the volume ratio of the solubilization buffer to the suspension buffer in (i) is about 1:1. In some embodiments, the volume ratio of the solubilizate from (i) to WFI is about 1:1. In some embodiments, the volume ratio of the folding buffer to the diluted solubilizate from (ii) is about 1:1. In some embodiments, the volume ratio of the folding mixture from (iii) to WFI is about 1:1. In some embodiments, the folding process of G-CSF at (b) includes: (i) incubating the solubilized G-CSF from (a) for a period of about 14-24 hours; (ii) performing a primary dilution of the incubated G-CSF from (i) at a volume ratio of about 1:1 with WFI; (iii) adding the folding buffer and incubating the diluted G-CSF mixture obtained from (ii) without mixing for a further period of about 20-24 hours; and (iv) performing a secondary dilution of the diluted G-CSF mixture obtained from (iii) at a volume ratio of about 1:1 with WFI.

In some embodiments, the incubation is carried out at a temperature of about 2° C. to about 25° C. In some embodiments, the incubation is carried out at a temperature of about 20±2° C. In some embodiments, the incubation is carried out at a temperature of about 4° C. In some embodiments, the primary dilution and/or secondary dilution is carried out by dripping the G-CSF-containing mixture into the WFI.

In some embodiments, the reduced form of a thiol redox pair is the reduced form of cysteine, glutathione, penicillamine, N-acetyl-penicillamine, 2-mercaptoacetic acid, 2-mercaptopropionic acid, 3-mercaptopropionic acid, mercaptosuccinic acid, mercaptopyruvate, mercaptoethoanol, monothioglycerol, γ-glutamylcysteine, cysteinylglycine, cysteamine, N-acetyl-L-cysteine, homocysteine, or lipoic acid (dihydrolipoamide). In some embodiments, the reduced form of a thiol redox pair is reduced glutathione (GSH). In some embodiments, the reduced form of a thiol redox pair in the folding buffer is cysteine. In some embodiments, the cysteine is present in the folding buffer at a concentration ranging from about 20 μM to 200 μM. In some embodiments, the cysteine is present in the folding buffer at a concentration of about 40 μM, about 50 μM, about 80 μM, or about 160 μM. In some embodiments, the folding buffer is added to the solubilizate to a final concentration of cysteine of about 80 μM.

In some embodiments, the recovery of the folded G-CSF includes one or more techniques selected from the group consisting of affinity chromatography, anion exchange chromatography (AEX), cation exchange chromatography (CEX), hydroxyapatite chromatography, size exclusion chromatography (SEC), hydrophobic interaction chromatography (HIC), metal affinity chromatography, mixed mode chromatography (MMC), centrifugation, diafiltration, and ultrafiltration. In some embodiments, the anion exchange chromatography includes DEAE Sepharose chromatography. In some embodiments, the cation exchange chromatography includes CM Sepharose chromatography. In some embodiments, the diafiltration and/or ultrafiltration includes a polyether sulfone membrane.

In some embodiments, the G-CSF is a human G-CSF (hG-CSF). In some embodiments, the G-CSF containing IBs are obtained from a recombinant cell expressing G-CSF wherein the expressed G-CSF forms the IBs in the cell. In some embodiments, the recombinant cell is a prokaryotic cell or a eukaryotic cell.

In some embodiments, the methods as disclosed herein do not include a strong denaturing agent, a strong reducing agent, a redox reaction, and/or a heavy metal. In some embodiments, the strong reducing agent is urea, tris-2-carboxyethylphoshpine.HCl (TCEP), or dithiothreitol (DTT). In some embodiments, the heavy metal is copper.

In one aspect, some embodiments of the disclosure relate to a granulocyte colony-stimulating factor (G-CSF) purified or isolated by a method disclosed herein.

In a related aspect, some embodiments of the disclosure relate to a pharmaceutical composition including a therapeutically effective amount of a G-CSF as disclosed herein, and a pharmaceutically acceptable auxiliary substance. In some embodiments, the pharmaceutical compositions is a liquid composition, a lyophilisate, or a powder.

In another aspect, some embodiments of the disclosure relate to a method for treating or preventing a disease in a subject including administering to the subject a therapeutically effective amount of a G-CSF as disclosed herein and/or a pharmaceutical composition as disclosed herein. In some embodiments, the disease is neutropenia.

### BRIEF DESCRIPTION OF THE DRAWINGS

FIG. 1 graphically illustrates the effects on protein yield and folding rates of G-CSF during solubilization and folding operation, as demonstrated for three non-limiting exemplary concentrations of denaturing agent in the solubilization buffer.

FIG. 2 shows a histogram illustrating the folding efficiency of G-CSF in the presence of cysteine during folding operation, as demonstrated for three non-limiting exemplary concentrations of cysteine in the folding buffer.

FIG. 3 shows a plot summarizing the results of an experiment performed to evaluate the effects of folding reaction time on the G-CSF product yield and quality in accordance with some embodiments of the disclosure.

FIG. 4 shows a plot summarizing the results of another experiment performed to evaluate the effects of folding time on the G-CSF product yield and quality in accordance with some embodiments of the disclosure.

FIG. 5 shows an overlay of the experimental data presented in FIG. 3 and FIG. 4, demonstrating the consistency of the methods disclosed herein.

### DETAILED DESCRIPTION OF THE DISCLOSURE

The present disclosure provides, inter alia, methods for the purification and/or preparation of granulocyte colony-stimulating factor (G-CSF) and compositions of G-CSF. The methods provide for improved production of G-CSF, (e.g., recombinant human G-CSF) from IBs produced in prokaryotic cells. The disclosure provides, inter alia, to methods of purifying G-CSF from IBs produced in a recombinant host cell such as E. coli, by solubilization of the G-CSF protein from the IBs in a buffer containing a denaturing agent, folding the solubilized G-CSF protein by diluting the solubilizate with a folding buffer containing only the reduced form of a thiol redox pair to obtain folded G-CSF, wherein the diluting step is carried out via a sequential stepwise dilution process. As described in greater detail below, the methods of the disclosure are particularly suitable for the preparation of biologically active G-CSF with improved purity and/or functional activity.

### Definitions

Unless otherwise defined, all terms of art, notations and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

The singular form “a”, “an”, and “the” include plural references unless the context clearly dictates otherwise. For example, the term “a cell” includes one or more cells, comprising mixtures thereof. “A and/or B” is used herein to include all of the following alternatives: “A”, “B”, “A or B”, and “A and B”.

The term “about”, as used herein, has its ordinary meaning of approximately. If the degree of approximation is not otherwise clear from the context, “about” means either within plus or minus 10% of the provided value, or rounded to the nearest significant figure, in all cases inclusive of the provided value. Where ranges are provided, they are inclusive of the boundary values.

It is understood that aspects and embodiments of the disclosure described herein include “comprising”, “consisting”, and “consisting essentially of” aspects and embodiments.

Headings, e.g., (a), (b), (i) etc., are presented merely for ease of reading the specification and claims. The use of headings in the specification or claims does not require the steps or elements be performed in alphabetical or numerical order or the order in which they are presented.

### Granulocyte Colony-Stimulating Factor (G-C Sf)

Granulocyte colony-stimulating factor (G-CSF) is a multifunctional cytokine which is widely used for treating neutropenia in humans. G-CSF is a hematopoietic lineage-specific cytokine mainly produced by fibroblasts and endothelial cells from bone marrow stroma and by immunocompetent cells (such as, e.g., monocytes, macrophages). The receptor for G-CSF (G-CSFR) is part of the cytokine and hematopoietin receptor superfamily and G-CSFR mutations cause severe congenital neutropenia. The main action of G-CSF/G-CSFR linkage is stimulation of the differentiation, proliferation, mobilization, survival, and chemotaxis of neutrophils in the bone marrow and control their release to the bloodstream. In addition, many other G-CSF effects have been reported, including growth and migration of endothelial cells, decrease of norepinephrine reuptake, increase in osteoclastic activity and decrease in osteoblast activity.

The therapeutic indications of G-CSF have been widely reported and include non-neutropenic patient infections, reproductive medicine, neurological disturbances, regeneration therapy after acute myocardial infarction and of skeletal muscle, and hepatitis C therapy. In oncology, G-CSF is utilized especially for the primary prophylaxis of chemotherapy-induced neutropenia, but it can be used for hematopoietic stem cell transplantation, wherein it can produce monocytic differentiation of some myeloid leukemias.

Human G-CSF (hG-CSF) can be produced in eukaryotic organisms (e.g., yeast and mammalian cell lines) and in prokaryotic organism such as bacteria (e.g., E. coli). The form of hG-CSF is produced depends on the type of host organism used for expression. Human G-CSF mRNAs contain coding sequences for a hydrophobic leader sequence typical of secreted proteins. When hG-CSF is expressed in eukaryotic cells, it is generally produced in a soluble form and secreted. On the other hand, when hG-CSF is produced in prokaryotic cells, the produce hG-CSF can be formed as intracellular compact aggregates called inclusion bodies with limited solubility. Generally, inclusion bodies formed in recombinant host cells have complex secondary structures and are densely aggregated and appear as bright spots under the microscope. It has been reported that the formation of IBs of recombinant proteins in host cells is one of the manifestations of protein misfolding, and that aggregation results from the accumulation of partially folded polypeptide chains that fail to reach the native conformation.

Functionally active G-CSF contains two intra-molecular disulfide bonds occurring between cysteine residues at position 36/42 and 64/74 that are believed to provide stability to the protein. The disulfide bonds in these molecules stabilize the structures and make them resistant to relatively harsh treatment (some proteases, high temperatures, denaturing solvents, extreme pH), which do lead to denaturation after reduction of disulfide bonds.

A number of purification processes are currently available for production of G-CSF from eukaryotic cells. Achieving protein secretion through extracellular eukaryotic organisms forms an entirely different technology in comparison with protein expression through intracellular prokaryotic organisms. In particular, several existing processes have been developed for soluble G-CSF expressed and secreted by recombinant eukaryotic cells (e.g., yeast cells and mammalian cells) into the culture medium. Therefore, these processes are not readily applicable to recombinant G-CSF expressed in E. coli or other host cells where G-CSF is produced in the form of inclusion bodies with limited solubility. For example, the high-level expression of eukaryotic proteins in prokaryotic cells such as E. coli often leads to formation of insoluble IBs in the cytoplasm.

Since aggregated polypeptides are usually misfolded (e.g., incorrect disulfide pairings) and therefore devoid of any biological activity, a key issue in product recovery is to reconstitute the correct three-dimensional conformation. Generally, multiple steps need to be taken to obtain a functional G-CSF protein in its correctly folded form from host cells in which it is accumulated in a denatured form, e.g., the biologically inactive, unfolded or predominantly misfolded form of the expressed G-CSF. Therefore, for G-CSF produced in E. coli cell, solubilization of IBs and folding of solubilized G-CSF are additional steps to be taken into account. In this case, bacterial cells carrying inclusion bodies generally need to be disintegrated, and the inclusion bodies harvested by, e.g., centrifugation or microfiltration, and then dissolved in a solubilization buffer. The denatured protein is then transferred into an environment that favors the recovery of its native conformation, wherein some or all of its native secondary and/or tertiary structure are restored. Before adopting its native conformation, the protein undergoes a transition through various semi-stable intermediates. Since intermediates in the early stages of the folding pathway have highly exposed hydrophobic domains, which are prone to associate, they tend to form aggregates. It has been reported that intramolecular interactions are concentration-independent, whereas intermolecular interactions are concentration-dependent. The higher the protein concentration, the higher the risk of intermolecular misfolding, and vice versa. To minimize aggregation, the protein concentration generally has to be kept low, which often is the bottleneck in industrial processes.

On a commercial scale, yield losses from a multi-step process can be highly significant. In addition, folding of recombinant proteins produced in the form of IBs often incorporates strong denaturing agents, strong reducing agents, a redox reaction, and/or heavy metals. Many of these agents have challenges including being costly at a large production scale and caustic in stainless steel manufacturing plants.

As described in greater detail below, the present disclosure provides improved methods for production of G-CSF which is particularly suitable for commercial production scale.

### Methods for Preparing G-CSF

Although it can often be difficult to recover the recombinantly produced G-CSF from IBs, the methods provided herein takes advantage of the accumulation of recombinant G-CSF within the host cells in an insoluble form for downstream processing that yields better recovery.

Generally, the recombinant protein product represents at least 40 to 50% of the total protein content of IBs. In addition, the protein aggregates can be relatively easily separated from the soluble components of lysed cells by centrifugation or microfiltration. Therefore, purification procedures for inclusion body proteins generally require fewer steps than procedures for comparable proteins expressed in soluble form, which tends to save time and reduce losses. The initial step in such purification procedures is the release of IBs from the cells, which generally involve cell disruption and separation of the insoluble IB material from soluble cellular components. Such processes are considered to be relatively simple. Cells can be lysed using mechanical techniques such as homogenization or by chemical or enzymatic methods. Soluble cellular materials can be removed from the inclusion body preparation by cycles of centrifugation and resuspension in buffer. Soluble materials can also be removed by filtration, which decreases the fixed and operating costs and is more amenable to scale-up. At the completion of this step, the resulting preparation contains essentially the IBs with a small amount of contaminating cell debris. Optionally, differential centrifugation in a sucrose gradient may be used to remove contaminants such as cell debris and membrane proteins. The purified inclusion body fraction can then be pelleted and stored for downstream processing.

Once the IBs have been isolated, they can be solubilized in the presence of denaturing agents and then further purified in the denatured state. An important step of the D3 protein purification scheme is the folding (e.g., renaturing and/or refolding) of the denatured protein to form a biologically active product. Although folding can be relatively simple for small monomeric proteins, this process can be quite complicated when the protein consists of more than one polypeptide chains or contains several disulfide bonds, such as G-CSF. Inadequate folding processes can result in overall low recovery yields of active protein.

In one aspect of the disclosure, some embodiments disclosed herein concern a method for the isolation and/or purification of G-CSF from IBs produced in a recombinant host cell such as E. coli, which includes dissolving the G-CSF protein from the IBs in a solubilization buffer containing a denaturing agent, followed by folding the solubilized G-CSF protein by diluting the solubilizate with a folding buffer containing only the reduced form of a thiol redox pair to obtain folded G-CSF, wherein the diluting step is carried out via a sequential stepwise dilution process.

In some embodiments, the G-CSF obtained according to the methods disclosed herein includes biologically active form of G-CSF, e.g., a form or molecule of G-CSF which is in a monomeric and non-denatured state and is capable of promoting the differentiation and proliferation of hematopoietic precursor cells and the activation of mature cells of the hematopoietic system. In some embodiments, the G-CSF obtained according to the methods disclosed herein includes biologically active G-CSF with improved purity and/or functional activity.

In some embodiments of the methods disclosed herein, the obtained G-CSF contains biologically active, correctly folded G-CSF with a purity of greater than 80%. In some embodiments, the obtained G-CSF includes biologically active, correctly folded G-CSF with a purity of greater than 85%, 90%, 95%, 96%, 97%, 98%, or 99%. Various methods for quantifying the degree of purification of the obtained G-CSF will be known to those of skill in the art in light of the present disclosure. These include, for example, determining the specific activity of an active G-CSF, or assessing the amount of G-CSF in the end product by SDS-PAGE analysis. An exemplary method for assessing the purity of a G-CSF obtained from the disclosed method is to calculate the specific activity of the obtained G-CSF, and to compare it to the specific activity of the initial extract, and to thus calculate the degree of purity.

The biological activity of the G-CSF obtained according to the present disclosure can be determined by a number of techniques known in the art, for example, by means of a bioassay known in the art and compared with the activity of a standard, commercially available G-CSF. For example, biological activity of the G-CSF obtained from the method as disclosed herein can be determined by an assay based on stimulation of cellular proliferation (NFS-60 cells) using the method described by Hammerling, U. et al. (J Pharm Biomed Anal 13, 9-20 (1995)) and the use of an international standard human recombinant G-CSF. In this assay the mouse cell line NFS-60, which is responsive to G-CSF can be cultivated in a suitable medium, such as RPMI 1640 culture medium, and supplemented with 2 mM glutamine, 10% FCS, 0.05 mM 2-mercaptoethanol and 60 ng/ml G-CSF. For the activity test, the cells are washed twice with medium without G-CSF, and placed in 96-well plates at a suitable concentration, e.g., of 2×10⁴ cells per well and incubated for three days at 37° C. and 4.5% CO2 with varying concentrations of the purified G-CSF and the standard, respectively. Subsequently, the cells can be stained with XTT reagent (Thermo Fischer Scientific) and the absorption at 450 nm is measured in a microtiter-plate reader.

It can then be shown that cells treated with the G-CSF purified or isolated as described herein grow just as well or better as those cells that are treated with the standard. In particular, it can be shown that purified G-CSF obtained according to the method of the present disclosure is characterized by a biological activity of 80-100% referring to WHO-reference standard in the NFS-60 proliferation assay.

In some embodiments, the G-CSF purified or isolated as described herein is correctly folded G-CSF with improved purity and/or functional activity. In some embodiments, the G-CSF as described herein has a purity of greater than 80% such as, for example, a purity of greater than 85%, 90%, 95%, 96%, 97%, 98%, or 99%. In some embodiments, the obtained G-CSF exhibits a significant increase in specific activity, for example, a specific activity of at least 1×10⁵ IU/mg. In some embodiments, the obtained G-CSF has a specific activity of at least 1×10⁶ IU/mg, preferably at least 1×10⁷ IU/mg, more preferably within a range of specific activity 2-9×10⁷ IU/mg, and most preferably a specific activity of about 1×10⁸ IU/mg, wherein the specific activity is measured by a method based on stimulation of cellular proliferation.

### Solubilization

The G-CSF contained in the IBs can be solubilized under denaturing conditions in a solubilization buffer containing one or more denaturing agents (also referred to as solubilizing agents), which are compounds having the ability to remove some or all of the G-CSF protein's secondary and/or tertiary structure when placed in contact with the G-CSF protein. During solubilization process, the G-CSF contained in the IBs becomes dissolved by treating with a denaturing agent whereby removing some or all of the G-CSF protein's secondary and/or tertiary structure. Intermolecular and intramolecular interactions, which are not present in the native G-CSF protein, become broken during solubilization process, thereby resulting in a monomeric dispersion of the G-CSF in the solubilization buffer. Denaturing agents suitable for use in the methods of the disclosure encompass agents that are able to unfold a protein, thus resulting in a reduction or loss of the native protein conformation. Exemplary denaturing agents suitable for use in the methods of the disclosure include, but are not limited to, mild denaturing agents, strong denaturing agents, an ionic detergents (e.g., cationic detergents and anionic detergents), or a combination thereof.

In some embodiments, the denaturing agent in the solubilization buffer includes a mild denaturing agent which is characterized by a detergent mechanism of action for denaturing proteins, e.g., through binding the protein chains and coating with surfactant molecules. Ionic detergent surfactants, including cationic, anionic, and zwitterionic detergent surfactants, are suitable hydrophilic surfactants for use in the methods of the present disclosure. Non-limiting examples of ionic detergent surfactants suitable for use in the solubilization buffer include, N-lauroyl sarcosine (sarkosyl), sodium dodecyl sulfate (SDS), sodium lauryl sulfate, polyoxyethylene poloxypropylene glycol, cocoamphoacetate, lithium dodecyl sulfate, sodium octyl sulfate, deoxycholic acid, sodium cholate hydrate, sodium deoxycholate, sodium glycocholate, sodium taurodeoxycholate sodium oleate, sodium lauryl sarcosinate, sodium dioctyl sulfosuccinate, sodium choleate, sodium taurocholate, lauroyl camitine, palmitoyl carnitine, myristoyl carnitine. In some embodiments, the mild denaturing detergent in the solubilization buffer includes an alkyl sulfate detergent. In some embodiments, the denaturing agent in the solubilization buffer includes N-lauroyl-sarcosine (i.e., NLS or sarcosyl), sodium dodecyl sulfate (SDS), or any combination thereof. In some embodiments, the denaturing agent in the solubilization buffer includes sarkosyl.

In some embodiments, the denaturing agent in the solubilization buffer includes a strong denaturing agent which is characterized by a chaotropic mechanism of action for denaturing proteins, e.g., by disrupting hydrogen bonding between water molecules and thus reducing protein stability. Non-limiting examples of strong denaturing agents suitable for use in the methods of the disclosure include, urea, guanidine hydrochloride (GndHCl), tris-2-carboxyethylphoshpine.HCl (TCEP), dithiothreitol (DTT), sodium thiocyanant, potassium thiocyanate, pH-extreme (diluted acidic or bases), strong denaturing detergents, salts (e.g., chloride, nitrates, thiocyanates, trichloroacetate), chemical derivatization (sulfitolyse, or reactions on the bases with citraconanhydrid), and solvents such as, e.g., 2-amino-2-methyl-1-propanol or alcohols, dimethyl sulfoxide (DMSO), and dimethyl sulfide (DMS).

In some embodiments of the methods disclosed herein, the solubilization buffer does not include a chaotropic agent. In some embodiments, the solubilization buffer does not include urea, GdmCl, sodium thiocyanate, potassium thiocyanate, mercaptoethanol, DTT, TCEP, dithiothreitol, or DMSO.

In some embodiments, the denaturing agent in the solubilization buffer includes an anionic detergent. Non-limiting examples of anionic detergents suitable for use in the methods and compositions disclosure herein include alkyl sulfates, alkyl sulfonates, and bile salts. Specific examples anionic detergents suitable for use in the present disclosure herein include, but are not limited to, lithium dodecyl sulfate, sodium octyl sulfate, sodium pentanesulfonate, sodium hexanesulfonate, 1-octanesulfonic acid sodium, 4-dodecylbenzenesulfonic acid, ethanesulfonic acid sodium salt monohydrate, sodium 1-butanesulfonate anionic detergent, sodium 1-decanesulfonate, sodium 1-heptanesulfonate, sodium 1-nonanesulfonate, sodium 1-octanesulfonate. Specific examples of bile salts suitable for use in the present disclosure include, but are not limited to, chenodeoxycholic acid, chenodeoxycholic acid diacetate methyl ester, cholic acid, deoxycholic acid, glycocholic acid, sodium chenodeoxycholate, sodium cholate hydrate, sodium choleate, sodium cholesteryl sulfate, sodium deoxycholate, sodium glycocholate, sodium glycochenodeoxycholate, sodium taurochenodeoxycholate, sodium taurolithocholate, sodium taurohyodeoxycholate. Additional anionic detergents suitable for use in the present disclosure include, but are not limited to, dicyclohexyl sulfosuccinate sodium, dihexadecyl phosphate, dihexyl sulfosuccinate sodium, docusate sodium, lithium 3,5-diiodosalicylate, N-lauroyl sarcosine sodium, N-lauroyl sarcosine (sarkosyl), sodium octanoate, and Triton™ QS-15.

In some embodiments, the anionic detergent in the solubilization buffer includes N-lauroyl-sarcosine (i.e., NLS or sarcosyl), sodium dodecyl sulfate (SDS), or any combination thereof. In some embodiments, the anionic detergent in the solubilization buffer includes sarkosyl. In some embodiments, sarkosyl is present in the solubilization buffer in an amount ranging from about 0.2% to about 5.0% by weight. For example, in some embodiments, the solubilization buffer contains sarkosyl in an amount ranging from about 0.2% to 5.0%, about 0.5% to 4.0%, about 1.0% to 3.0%, about 1.5% to 2.0%, about 0.2% to 3.0%, about 0.5% to 2.0%, about 1.0% to 2.0% by weight. In some embodiments, the solubilization buffer contains sarkosyl in an amount of about 0.2%, 0.5%, 1.0%, 1.5%, 2.0%, 2.5%, 3.0%, 3.5%, 4.0%, 4.5, or 5.0% by weight. In some embodiments, the solubilization buffer contains sarkosyl in an amount of about 0.2% by weight. In some embodiments, the solubilization buffer contains sarkosyl in an amount of about 0.56% by weight. In some embodiments, the solubilization buffer contains sarkosyl in an amount of about 1.0% by weight. In some embodiments, the solubilization buffer contains sarkosyl in an amount of about 2.0% by weight.

Examples of buffering agents suitable for use in the solubilization buffer include, but are not limited to, tris(hydroxymethyl)aminomethane (Tris), phosphate, citrate, acetate, succinate, MES, MOPS, or ammonium and their salts or derivatives thereof. In some embodiments, the suspension buffer includes Tris as a buffering agent. In some embodiments, the solubilization buffer includes Tris with a molarity within the range of about 20 mM to 60 mM, such as for example, about 20 mM to 40 mM, about 30 mM to 50 mM, about 40 mM to 60 mM, about 20 mM to 30 mM, about 30 mM to 60 mM, and about 40 mM to 50 mM. In some embodiments, the solubilization buffer includes Tris with a molarity of about 20 mM, 30 mM, 40 mM, 50 mM, or 60 mM. In some embodiments, the solubilization buffer includes Tris with a molarity of about 40 mM. In some embodiments, the Tris molarity in the solubilization buffer is similar to the Tris molarity in the suspension buffer. In some embodiments, the Tris molarity in the solubilization buffer is different from the Tris molarity in the suspension buffer.

In some embodiments of the disclosure, suitable pH for the solubilization buffer ranges from about 7.5 to 9.0, such as for example, from about 7.5 to 8.0, about 8.0 to 8.5, about 8.5 to 9.0, about 7.5 to 8.5, about 8.0 to 9.0. The pH range is chosen to optimize the solubilization of the IBs and to preserve the desired characteristics of the G-CSF contained in the IBs. In some embodiments, the solubilization buffer has a pH of about 7.5, 7.6, 7.7, 7.8, 7.9, or 8.0. In some embodiments, the solubilization buffer has a pH of about 8.1, 8.2, 8.3, 8.4, 8.5, 8.6, 8.7, 8.8, 8.9, or 9.0. In some embodiments, the solubilization buffer has a pH of about 8.4. In some embodiments, the solubilization buffer includes 40 mM Tris at pH of 8.4.

In some embodiments, the IBs containing G-CSF are suspended in a suspension buffer to form an inclusion bodies suspension prior to solubilization. Examples of buffering agents suitable for use in the suspension buffer include, but are not limited to, tris(hydroxymethyl)aminomethane (Tris), phosphate, citrate, acetate, succinate, MES, MOPS, or ammonium and their salts or derivatives thereof. In some embodiments, the suspension buffer includes Tris as a buffering agent. In some embodiments, the suspension buffer includes Tris with a molarity within the range of about 20 mM to 60 mM, such as for example, about 20 mM to 40 mM, about 30 mM to 50 mM, about 40 mM to 60 mM, about 20 mM to 30 mM, about 30 mM to 60 mM, and about 40 mM to 50 mM. In some embodiments, the suspension buffer includes Tris with a molarity of about 20 mM, 30 mM, 40 mM, 50 mM, or 60 mM. In some embodiments, the suspension buffer includes Tris with a molarity of about 40 mM,

In some embodiments of the disclosure, suitable pH for the suspension buffer ranges from about 7.0 to 8.0, such as for example, from about 7.0 to 7.5, about 7.2 to 7.6, about 7.3 to 7.7, about 7.4 to 7.8, about 7.5 to 7.9. In some embodiments, the suspension buffer has a pH of about 7.0, 7.1, 7.2, 7.3, 7.4, 7.5, 7.6, 7.7, 7.8, 7.9, or 8.0. In some embodiments, the suspension buffer has a pH of about 7.6. In some embodiments, the suspension buffer includes 40 mM Tris at pH of 7.6.

In order to ensure that the IBs containing G-CSF are efficiently and maximally suspended, an appropriate ratio of IBs and suspension buffer is required. According to some embodiments of the present disclosure, 10 g to 100 g of suspension buffer per gram of IBs (e.g., pellet mass) are used, for example from about 20 g to 90 g, about 30 g to 80 g, about 40 g to 70 g, about 50 g to 60 g of suspension buffer per gram of IBs. In some embodiments, about 10 g to 50 g, about 20 g to 60 g, about 30 g to 70 g, about 40 g to 80 g, about 50 g to 90 g of suspension buffer per gram of IBs are used. In some embodiments, about 10 g, 20 g, 30 g, 40 g, 50 g, 60 g, 70 g, 80 g, 90 g, or 100 g of suspension buffer per gram of IBs are used. In some embodiments, about 25 g of suspension buffer per gram of IBs are used.

In some embodiments, the pH of the suspension buffer is similar to the pH of the solubilization buffer. In some embodiments, the pH of the suspension buffer is different from the pH of the solubilization buffer. In some embodiments, the volume ratio of the suspension buffer to the solubilization buffer is adjusted such that the final pH of the solubilizate is about 7.6 to about 8.4, for example, about 7.6, 7.7, 7.8, 7.9, 8.0, 8.1, 8.2, 8.3, or 8.4. In some embodiments, the volume ratio of the suspension buffer to the solubilization buffer is adjusted such that the final pH of the solubilizate is about 7.6. In some embodiments, the volume ratio of the suspension buffer to the solubilization buffer is adjusted such that the final pH of the solubilizate is about 7.8 to about 8.2, for example, about 7.8, 7.9, 8.0, 8.1, or 8.2. In some embodiments, the volume ratio of the suspension buffer to the solubilization buffer is adjusted such that the final pH of the solubilizate is about 8.0.

In some embodiments of the methods disclosed herein, the solubilization buffer includes about 40 mM Tris, about 2% sarkosyl, at pH of about 8.4. In some embodiments of the methods disclosed herein, the solubilization buffer includes 40 mM Tris, 2% sarkosyl, at pH of 8.4.

In order to ensure an efficient and complete solubilization of the suspended IBs, an appropriate ratio of IBs (and solubilization buffer is required). According to some embodiments of the present disclosure, 10 g to 50 g of solubilization buffer per gram of IBs (lysed pellet mass) are used, for example from about 10 g to 30 g, about 15 g to 35 g, about 20 g to 40 g, about 30 g to 45 g of solubilization buffer per gram of IBs. In some embodiments, about 10 g to 45 g, about 20 g to 35 g, about 25 g to 30 g, about 30 g to 50 g, about 25 g to 45 g of solubilization buffer per gram of IBs are used. In some embodiments, about 10 g, 15 g, 20 g, 25 g, 30 g, 35 g, 40 g, 45 g, or 50 g of solubilization buffer per gram of IBs are used. In some embodiments, about 25 g of solubilization buffer per gram of IBs are used.

In some embodiments, 10 mL to 100 mL of solubilization buffer per gram of IBs (lysed pellet mass) are used, for example from about 20 mL to 90 mL, about 30 mL to 80 mL, about 40 mL to 70 mL, about 50 mL to 60 mL of solubilization buffer per gram of IBs. In some embodiments, about 10 mL to 50 mL, about 20 mL to 60 mL, about 30 mL to 70 mL, about 40 mL to 80 mL, about 50 mL to 90 mL of solubilization buffer per gram of IBs are used. In some embodiments, about 10, 20, 30, 40, 50, 60, 70, 80, 90, or 100 mL of solubilization buffer per gram of IBs are used.

In some embodiments disclosed herein, to ensure an efficient and complete solubilization of the suspended IBs, the solubilization time of the methods of the disclosure generally ranges from about 4 hours to 48 hours. In some embodiments, after addition of the solubilization buffer to the inclusion bodies suspension, the solubilization mixture is incubated for a time period ranging from about 4 hours to 48 hours. In some embodiments, the solubilization time ranges from about 4 to 42 hours, about 12 to 36 hours, or about 18 to 30 hours. In some embodiments, the solubilization time ranges from about 4 to 24 hours, about 12 to 30 hours, about 18 to 36 hours, about 24 to 42 hours, or about 30 to 48 hours. In some embodiments, the solubilization time is about 4 to 24 hours. In some embodiments, the solubilization time is about 14 to 24 hours.

### Folding

An important step in the process of isolating and/or purifying G-CSF produced in IBs is the folding of the denatured protein (e.g., renaturation) to form a biologically active product. In theory, renaturation may be accomplished by removal of the denaturing agent. However, in practice, the folding process is more complex, and suboptimal renaturation can often lead to protein aggregation and/or inactivation with a low recovery of correctly folded protein. Without being bound to any particular theory, the degree of protein aggregation is dependent on environmental parameters. Often aggregation is reduced when the pH of the medium is far removed from the isoelectric point of the protein. However, the relations between the solution pH and the degree of protein aggregation are much more complex. Aggregation generally increases with increasing temperature due in part to an increased probability of collision between protein molecules at elevated temperatures. One of the factors considered to be important for maximizing folding yield is the rate of denaturing agent removal. The removal of the denaturing agents can be accomplished by a variety of techniques. These include dilution, dialysis, gel filtration, diafiltration and immobilization on a solid support. In some embodiments, the removal of the denaturing agents can be accomplished by dilution. Among the known protein folding strategies, dilution is generally considered one of the simplest methodologies. In industrial scale applications, dilution is often used for folding of recombinant proteins expressed in host cells as IBs. In several existing methods for isolating G-CSF, dilution generally is carried out in one step by mixing/diluting the solution containing solubilized protein with a diluent containing a denaturing agent in an amount necessary to reach the optimal level of dilution. When the concentration of denaturing agent is below a certain threshold level, the recombinant protein start to regain its biologically active three-dimensional conformation. Depending on the chosen folding conditions, folding begins within milliseconds to seconds. However, in this initial burst phase, the recombinant protein is highly susceptible to aggregation. To minimize aggregation, the protein concentration generally has to be kept low.

In contrast, as an important distinguishing feature relative to existing processes for the purification of G-CSF produced in IBs, some embodiments of the methods disclosed herein involve a process of stepwise dilution. As described in greater detail below, in some embodiments of the disclosure, the removal of the denaturing agent(s) from the solubilizate, can be achieved by gradually reducing the denaturing agent concentration in the solubilizate in a stepwise dilution procedure. This is one of the key features of the disclosed methods because one of the factors considered to be important for maximizing folding yield is the rate of denaturing agent removal.

In some embodiments of the methods disclosed herein, after solubilization step, the folding of the solubilized G-CSF can be achieved by diluting, via a sequential stepwise dilution process, the solubilizate within a folding buffer containing only the reduced form of a thiol redox pair, e.g. which can be referred to as thiol redox couple or thiol redox system, to initiate folding and to obtain folded G-CSF. In some embodiments of the methods disclosed herein, the sequential stepwise dilution process includes gradually reducing the denaturing agent concentration in the solubilizate. In some embodiments, the process of gradually reducing the denaturing agent concentration includes one or more of the following: (i) mixing the solubilization buffer with the suspension buffer in which the IBs containing G-CSF are suspended; (ii) diluting the solubilizate from (i) with WFI to form a diluted solubilizate; (iii) adding the folding buffer to the diluted solubilizate from (ii); and (iv) further diluting the folding mixture from (iii) with WFI. As discussed above, the multi-step folding process as disclosed herein can be advantageously implemented in industrial scale applications.

In some embodiments, the reduction in denaturing agent concentration is achieved by addition of the suspension buffer to the solubilization buffer. In some embodiments, the reduction in denaturing agent concentration is achieved by slow addition of the suspension buffer, by dripping, to the solubilization buffer until reaching a desired ratio of the suspension buffer to the solubilization buffer. In some embodiments, the volume ratio of the suspension buffer to the solubilization buffer is about 1:1, 1:2, 1:3, 1:4, 1:5, 5:1, 4:1, 3:1, or 2:1. In some embodiments, the volume ratio of the suspension buffer to the solubilization buffer in (i) is about 1:1. In some embodiments, the denaturing agent concentration is further reduced by addition of WFI to the solubilizate in (i). In some embodiments, the reduction in denaturing agent concentration is achieved by slow addition of the WFI, for example by dripping, to the solubilizate until reaching a desired ratio of the solubilizate to the WFI. In some embodiments, the volume ratio of the solubilizate from (i) to WFI is about 1:1, 1:2, 1:3, 1:4, 1:5, 5:1, 4:1, 3:1, or 2:1. In some embodiments, the volume ratio of the solubilizate from (i) to WFI is about 1:1.

In some embodiments, the denaturing agent concentration is further reduced by addition of the folding buffer to the diluted solubilizate from (ii). In some embodiments, the reduction in denaturing agent concentration is achieved by slow addition of the folding buffer, for example by dripping, to the diluted solubilizate until reaching a desired ratio of the folding buffer to the diluted solubilizate. In some embodiments, the volume ratio of the folding buffer to the diluted solubilizate from (ii) is about 1:1, 1:2, 1:3, 1:4, 1:5, 5:1, 4:1, 3:1, or 2:1. In some embodiments, the volume ratio of the folding buffer to the diluted solubilizate from (ii) is about 1:1. In some embodiments, the denaturing agent concentration is further reduced by addition of WFI to the folding mixture from (iii). In some embodiments, the reduction in denaturing agent concentration is achieved by slow addition of WFI, for example by dripping, to the folding mixture until reaching a desired ratio of the folding mixture to WFI. In some embodiments, the volume ratio of the folding mixture from (iii) to WFI is about 1:1, 1:2, 1:3, 1:4, 1:5, 5:1, 4:1, 3:1, or 2:1. In some embodiments, the volume ratio of the folding mixture from (iii) to WFI is about 1.1.

Generally, the methods for the isolation and/or preparation of G-CSF of the disclosure can be conducted at a temperature of about 2° C. to about 25° C., such as, for example, about 4° C. to about 15° C., about 10° C. to about 25° C., about 15° C. to about 25° C., about 20° C. to about 25° C., about 15° C. to about 20° C., or about 10° C. to about 15° C. In some embodiments, the methods can be conducted at a temperature of about 4±2° C. In some embodiments, the methods can be conducted at ambient temperatures, e.g., generally above 10° C. and advantageously at room temperature, i.e. at 20±2° C. The ambient temperature may vary between 10° C. and 30° C. and is preferably between 15° C. and 25° C., more preferably between 17° C. and 23° C. and particularly preferred between 19° C. and 21° C. In some embodiments, the methods of the disclosure are carried out at a temperature of about 15° C. to about 25° C. In some embodiments, the method of the disclosure are carried out at a temperature of about 20±2° C. In some embodiments, the suspending of the IBs, the solubilization of the suspended IBs, and the folding of the G-CSF are carried out at different temperatures. In some embodiments, the suspending of the IBs, the solubilization of the suspended IBs, and the folding of the G-CSF are carried out at the same temperature. In some embodiments, the folding of the G-CSF is carried out at a temperature of about 2° C. to about 25° C., such as, for example, about 4° C. to about 15° C., about 10° C. to about 25° C., about 15° C. to about 25° C., about 20° C. to about 25° C., about 15° C. to about 20° C., or about 10° C. to about 15° C. In some embodiments, the folding of the G-CSF is carried out at a temperature of about 4±2° C. In some embodiments, the folding of the G-CSF is carried out at a temperature of about 4° C. In some embodiments, the folding of the G-CSF is carried out at a temperature of about 20±2° C. In some embodiments, the folding of the G-CSF is carried out at a temperature of about 20° C.

It has been documented that the rate of folding of different proteins can vary from less than one second to several hours and even days. This is because the isomerization of disulfide bonds to form the correct cysteine pairs that are present in the native protein is slow and represents an important rate limiting step in folding. For this reason, the in-vitro folding of polypeptides containing several cysteine residues (such as G-CSF) is usually very slow and inefficient. According to some embodiments disclosed herein, to ensure an efficient and maximal folding of the solubilized G-CSF, the folding reaction time of the methods of the disclosure generally ranges from about 12 hours to 48 hours, which is generally dependent on the temperature of the folding process. In some embodiments, after addition of the folding buffer to the solubilizate, the folding mixture is incubated for a time period ranging from about 12 hours to 48 hours. In some embodiments, the folding reaction time ranges from about 12 to 24 hours, about 18 to 30 hours, about 24 to 36 hours, or about 30 to 48 hours. In some embodiments, the folding reaction time ranges from about 12 to 30 hours, about 18 to 36 hours, about 24 to 48 hours, about 30 to 48 hours, or about 18 to 24 hours. In some embodiments, the folding reaction time is about 16 to 24 hours. In some embodiments, the folding reaction time is about 20 to 24 hours.

In some embodiments, the folding process of G-CSF at (b) is a multi-step process and includes: (i) incubating the solubilizate containing G-CSF from (a) for a period of about 14-24 hours; (ii) performing a primary dilution of the incubated G-CSF from (i) at a volume ratio of about 1:1 with WFI; (iii) adding the folding buffer and incubating the diluted G-CSF mixture obtained from (ii) without mixing for a further period of about 20-24 hours; and (iv) performing a secondary dilution of the diluted G-CSF mixture obtained from (iii) at a volume ratio of about 1:1 with WFI.

In some embodiments, the primary dilution and/or secondary dilution is carried out by slowly adding the solubilizate and/or the G-CSF-containing mixture to WFI. In some embodiments, the method of the disclosure is performed in a continuous mode. In some embodiments, the primary dilution and/or secondary dilution is carried out by continuously feeding the G-CSF-containing mixture to a vessel containing WFI. By running the folding process continuously, time consumption and costs can be reduced and the yield of folded protein can be increased as compared to known methods. The method of the disclosure ensures, in particular in its continuous mode, fast and efficient processing of D3 proteins, thereby reducing inadvertent variations, such as variations in folding efficiency or product homogeneity.

In some embodiments, it is advantageous to carry out the dilutions in a mixing vessel suitably equipped for temperature-controlled operations to exclude any, even minimal aggregation. In some embodiments, the mixing vessel is thermally coupled with a cooling supply or refrigeration device. Mixing vessels suitable for use in the method of the disclosure are any mixers that ensure fast mixing and short mixing times, e.g. tubular jet mixers or static mixers commercially available. Such devices can be used to achieve the desired mixing efficiency. In the case that the mixer is a high-throughput continuous flow device, accurate control of the flows is of particular importance. With such mixers, mixing times as low as a few milliseconds on the small scale or a few seconds on the large scale can be achieved. In some embodiments, the mixing vessel comprises a dripping mechanism configured to enable dripping of the solubilizate and/or the G-CSF-containing mixture directly into a container comprising WFI (or vice versa), wherein the dripping rate can be adjusted or set within a desired range.

In some embodiments, the primary dilution is carried out by slow addition of the incubated solubilizate to WFI until reaching an end volume ratio of about 1:1. In some embodiments, the primary dilution is carried out by slow addition of WFI to the incubated solubilizate until reaching an end volume ratio of about 1:1. In some embodiments, the primary dilution step is carried out by a dripping mechanism. In some embodiments, the dripping rate is adjusted or set within a desired range.

In some embodiments, the secondary dilution is carried out by slow addition of the diluted G-CSF mixture to WFI until reaching an end volume ratio of about 1:1. In some embodiments, the secondary dilution is carried out by slow addition of WFI to the diluted G-CSF mixture until to reach an end volume ratio of about 1:1. In some embodiments, the primary dilution step is carried out by a dripping mechanism. In some embodiments, the dripping rate is adjusted or set within a desired range.

In some embodiments, the primary dilution and/or secondary dilution is carried out by slowly dripping the G-CSF-containing mixture into a vessel containing WFI. In some embodiments, the primary dilution and/or secondary dilution is carried out by continuously dripping the G-CSF-containing mixture into a vessel containing WFI.

In some embodiments, the methods disclosed herein include implementation of another important distinguishing feature relative to existing processes for the purification of G-CSF produced in IBs. In order to promote folding after solubilization step, existing purification processes often incorporate a folding buffer containing a thiol redox pair or thiol redox couple, e.g., a mixture of reduced and oxidized thiol agents. Thiol redox pairs commonly used in existing purification process of G-CSF are reduced and oxidized glutathione (GSH/GSSG), cysteine/cystine, cysteamine/cystamine, dithiothreitol (DTT)/GSSG, and dithioerythritol (DTE)/GSSG). Previous protein purification studies have reported that (i) when cysteine residues are present in the primary amino acid sequence of the recombinant protein, it is necessary to accomplished the folding in a redox environment which allows correct formation of disulfide bonds, and (ii) the use of a redox system of disulfide and thiol (e.g., reduced and oxidized glutathione) allows the correct formation of disulfide bonds in the target protein by promoting the rapid transformation of incorrectly formed disulfide bonds.

In contrast, as an important distinguishing feature relative to existing processes for the purification of G-CSF produced in IBs, some embodiments of the methods disclosed herein include a folding buffer which does not contain any oxidized thiol agent. In particular, in some embodiments of the methods disclosed herein, the folding buffer contains only a reduced form (e.g., reduced thiol agent) of a thiol redox pair. In some embodiments, the folding buffer contains one reduced thiol agent. In some embodiments, the folding buffer contains two reduced thiol agents. Yet in some other embodiments, the folding buffer contains three different reduced thiol agents. Reduced thiol agents suitable for use in the folding buffer of the methods disclosed herein include, but are not limited to, the reduced forms of cysteine, glutathione, penicillamine, N-acetyl-penicillamine, 2-mercaptoacetic acid, 2-mercaptopropionic acid, 3-mercaptopropionic acid, mercaptosuccinic acid, mercaptopyruvate, mercaptoethoanol, monothioglycerol, γ-glutamylcysteine, cysteinylglycine, cysteamine, N-acetyl-L-cysteine, homocysteine, or lipoic acid (dihydrolipoamide). In some embodiments, the reduced thiol agent in the folding buffer is the reduced form of the GSH/GSSG thiol redox pair (i.e., GSH; see also, Table 1 and Examples 1-2). In some embodiments, the only reduced thiol agent present in the folding buffer is GSH. In some embodiments, folding buffer which does not contain the oxidized form of glutathione (GSSG or glutathione disulfide). In some embodiments, the folding buffer of the disclosure contains only the reduced form of the redox cysteine/cystine thiol redox pair. In some embodiments, folding buffer which does not contain the oxidized form of redox cysteine/cystine thiol redox pair.

In some embodiments, the only reduced thiol reagent present in the folding buffer is cysteine (see also, Table 1 and Examples 1-2). Without being bound to any particular theory, it is believed that the cysteine present in the folding buffer promotes formation of disulfide bonds. In some embodiments, the cysteine is present in the folding buffer at a concentration ranging from about 20 μM to about 200 μM. In some embodiments, the cysteine is present in the folding buffer at a concentration of about 40 μM, about 50 μM, about 80 μM, or about 160 μM.

One skilled in the art will appreciate that the volume of the folding buffer added to the solubilizate can be adjusted such that a predefined final concentration of the reduced thiol reagent in the mixture can be obtained. In some embodiments, the folding buffer is added to the solubilizate to a final concentration of cysteine ranging from about 20 μM to about 200 μM. In some embodiments, the folding buffer is added to the solubilizate to a final concentration of cysteine of about 40 μM, about 50 μM, about 80 μM, or about 160 μM. In some embodiments, the folding buffer is added to the solubilizate to a final concentration of cysteine of about 80 μM.

Recovery

In some embodiments, the methods of the disclosure further include a process of recovering the G-CSF obtained from the folding step. The recovery of the G-CSF can be achieved by essentially separating the G-CSF from undesirable impurities present in the expression/processing system, such as host cell debris, aggregated unfolded protein, dimers, multimers and/or unfolded protein of G-CSF which should not be present in the intermediate or final product. The term “impurity” as used herein, in the broadest sense, to refer to a substance which differs from the biologically active molecule of G-CSF such that the biologically active molecule of G-CSF is not pure. The impurity can include host cell substances such as nucleic acids, lipids, polysaccharides, proteins, etc.; culture medium, and additives which are used in the preparation and processing of G-CSF. In some embodiments, the impurity can include at least one substance selected from the group consisting of biologically inactive monomeric forms, incorrectly folded molecules of G-CSF, oligomeric and polymeric forms of G-CSF, denatured forms of G-CSF, and host cell proteins. It will be understood by one having ordinary skill in the art that a denatured form of a recombinant protein of interest generally includes the biologically inactive, unfolded, or predominantly misfolded form of the recombinant protein obtained as a product of the recombinant production process. In some embodiments, the G-CSF recovered by the method according to the present disclosure may be commercialized in the bulk form obtained directly from the recovery step or further purified and/or formulated into specific formulations, e.g. into pharmaceutical compositions and formulations.

In some embodiments, the recovery of the folded G-CSF can include one or more chromatography techniques. Suitable examples of chromatography techniques include, but are not limited to, affinity chromatography, anion exchange chromatography (AEX), cation exchange chromatography (CEX), hydroxyapatite chromatography, size exclusion chromatography (SEC), hydrophobic interaction chromatography (HIC), metal affinity chromatography, and mixed mode chromatography (MMC). Non-chromatography separation techniques can also be considered, such as precipitation with salt, acid, or with a polymer PEG. Other non-limiting non-chromatography separation techniques suitable for the disclosed methods include centrifugation, extraction, dialysis, diafiltration, and ultrafiltration.

With regard to filtration techniques, it is often advantageous to subject the folded protein to a filtration step prior further processing in order to remove high-molecular particles, which are often protein aggregates formed during folding. In some embodiments of the methods of the disclosure, the folded protein solution is filtered through a filter cascade, for example, through a 10 μm and 1.2 μm filter cascade. Following filtration, the folded protein solution can be stored at appropriate conditions for downstream applications.

In some embodiments, the recovery process of the methods disclosed herein includes one or more of ultra-, micro- or diafiltration operation to remove contaminants such as cell debris, insoluble contaminating proteins, and nucleic acid precipitates. These filtration operation provides a convenient means to economically and efficiently remove cell debris, contaminating proteins and precipitate. It will be appreciated by one of ordinary skill in the art that in choosing a filter or filter scheme, it is important to ensure a robust performance in the event upstream changes or variations occur. Care should be taken to maintain the balance between good clarification performance and step yield. Suitable filter types can utilize cellulose filters, regenerated cellulose fibers, cellulose fibers combined with inorganic filter aids (e.g., diatomaceous earth, perlite, fumed silica), cellulose fibers combined with inorganic filter aids and organic resins, or any combination thereof, and polymeric filters to achieve effective removal. Suitable examples of polymeric filters include but are not limited to nylon, polypropylene, polyether sulfone. In some embodiment, the filtration operation, e.g., the diafiltration step and/or ultrafiltration step is performed using a polyether sulfone membrane. In some embodiment, the diafiltration step and/or ultrafiltration step is performed using a Sius Hystream membrane.

In some embodiments, one or more steps of ion exchange chromatography can be carried out after the folded G-CSF is ultrafiltrated and/or diafiltrated. The ion exchange chromatography can be carried out using single ion-exchange chromatography in order to remove other contaminants such as host cell proteins, in particular endotoxins and host cell DNA. In principle, cation exchange chromatography (CEX) and/or anion exchange chromatography (AEX) can be suitably used.

In some embodiments, the one or more ion exchange steps includes an AEX followed by a CEX. In these instances, the finding that AEX can be used in a non-binding mode (G-CSF in flow through) while contaminants such as residual denaturing agent, host cell proteins, or DNA bind to the resin, necessitates a two-step ion exchange chromatography in the order of AEX followed by CEX.

Generally, the AEX step can be performed by using any one of functional groups known for AEX chromatography of proteins. These groups include diethylaminoethyl (DEAE), trimethylaminoethyl (TMAE), quaterny aminomethyl (Q), and quaterny aminoethyl (QAE). These are commonly used functional anion exchange groups for biochromatographic processes. Suitable commercially available products include, for example, DEAE-Sepharose FF, DEAE-Sepharose CL-4B, Q-Sepharose FF, Q-Sepharose CL-4B, Q-Sepharose HP, Q-Sepharose XL, Q-Sepharose Big Beads, QAE-Sephadex, DEAE-Sephadex, Capto DEAE, Capto Q, Capto Q ImpRes, Source 15Q, Source 30Q, DEAF Sephacel. Macro-Prep High Q, Macro-Prep DEAF, Nuvia Q, TOYOPEARL DEAE-650, TOYOPEARL SuperQ-650, TOYOPEARL QAE-550, Fractogel EMD DEAE, Fractogel EMD TMAE, Biosepra Q Ceramic HyperD, and Biosepra DEAE Ceramic HyperD. In some particular embodiments, an AEX step with DEAE Sepharose FF is performed that allows particularly high flow rates and good product recovery.

In some embodiments of the methods disclosed herein, a CEX step with a selected material (e.g., CM Sepharose® FF) is performed that allows particularly high flow rates and good product recovery. In these instances, due to the fact that it is positively charged in an acidic environment, G-CSF is a strong binder and can be eluted with a linear sodium chloride gradient at an acidized pH in a small volume at a high concentration in the desired buffer. Systems and methods for performing cation exchange chromatography are well known to the person skilled in the art. Generally, the G-CSF binds to the cation exchange matrix within a specific pH range due to its positive total charge, while most of the contaminating substances like nucleic acids, lipopolysaccharides and proteins originating from host cells as well as ionic isomers of G-CSF and altered forms of G-CSF having different pH values are not capable of binding and appear in the flow-through or are of being removed by means of washing.

Suitable functional groups used for CEX resins include, but are not limited to, carboxymethyl (CM), sulfonate (S), sulfopropyl (SP) and sulfoethyl (SE). These are commonly used cation exchange functional groups for biochromatographic processes. Suitable commercially available products include, but are not limited to, carboxymethyl (CM) cellulose, AG 50 W, Bio-Rex 70, carboxymethyl (CM) Sephadex, sulfopropyl (SP) Sephadex, carboxymethyl (CM) sepharose CL-6B, CM sepharose HP, Hyper D-S ceramic (Biosepra) and sulfonate (S) Sepharose, SP Sepharose FF, SP Sepharose HP, SP Sepharose 15 XL, CM Sepharose FF, TSK gel SP 5PW, TSK gel SP-5PW-HR, Toyopearl SP-650M, Toyopearl SP-650S, Toyopearl SP-650C, Toyopearl CM-650M, Toyopearl CM-650S etc. Sulfopropyl matrices, in particular the products SP Sepharose XL and SP Sepharose FF (Fast Flow) and S-Sepharose FF. In some embodiments, the cation exchange material is a sulfopropyl cation exchange material. In some particular embodiments of the present disclosure, the CEX is performed with CM-Sepharose FF.

In some embodiments disclosed herein, in order to achieve higher product concentration of the G-CSF preparation obtained after folding process, the folded G-CSF protein can be dialyzed or diafiltrated to remove contaminants such as unwanted buffer components. In particular, diafiltration is a fractionation process of washing smaller molecules through a membrane, leaving the larger molecule of interest in the retentate. It is widely considered a convenient and efficient technique for removing or exchanging salts, removing detergents, separating free from bound molecules, removing low molecular weight materials, or rapidly changing the ionic or pH environment. The diafiltration process generally employs a microfiltration or an ultrafiltration membrane in order to remove a product of interest from slurry while maintaining the slurry concentration as a constant.

As described above, G-CSF proteins, e.g., human G-CSF, can be recombinantly produced in eukaryotic organisms (e.g., yeast and mammalian cell lines) or in prokaryotic organism such as bacteria (e.g., E. coli). The form of G-CSF is produced depends on the type of host organism used for expression. When the G-CSF is expressed in eukaryotic cells, it is generally produced in a soluble form and secreted. When G-CSF is produced in prokaryotic cells, the product is formed as inactive IBs, which generally have a secondary structure and are densely aggregated. In some embodiments of the present disclosure, the G-CSF is a human G-CSF (hG-CSF). In some embodiments, the IBs containing G-CSF are derived from a recombinant cell expressing G-CSF wherein the expressed G-CSF forms the IBs in the cell. In some embodiments, the recombinant cell is a prokaryotic cell or a eukaryotic cell.

In some embodiments, one or more of the buffers, for example the solubilization buffer, in the methods disclosed herein contains a reducing agent (e.g., reductant) in addition to the denaturing agent. The reducing agent can be included as a means to reduce exposed residues that have a propensity to form covalent intra- or intermolecular protein bonds and minimize non-specific bond formation. Suitable reducing agents are reduced glutathione (GSH), DTT, dithioerythritol (DTE), cysteine, β-mercaptoethanol, and monothioglycerol. In some embodiments, the reducing agent in the methods disclosed herein is DTT. In some embodiment of the present disclosure, the concentration of the reducing agent in the solubilization buffer is 1 to 100 milimol/L, preferably 1 to 10 milimol/L.

In some other embodiments, the methods disclosed herein do not include a strong denaturing agent, a strong reducing agent, a redox reaction, and/or a heavy metal. In some embodiments, one or more of the buffers (e.g., suspension buffer, the solubilization buffer, and folding buffer) in the methods disclosed herein does not contain a reducing agent. In some embodiments, one or more of the buffers in the methods disclosed herein does not contain urea, tris-2-carboxyethylphoshpine.HCl (TCEP), and/or DTT. In some embodiments, one or more of the buffers in the methods disclosed herein does not contain a heavy metal or a salt thereof. In some embodiments, one or more of the buffers in the methods disclosed herein does not contain the heavy metal copper or a salt thereof. In some embodiments, one or more of the buffers in the methods disclosed herein does not contain CuSO₄.

As discussed above, folding buffers containing thiol redox agents have been shown to be critical factor for facilitating renaturation and correct folding of proteins recombinantly produced in various host cells. Most common thiol redox agents used for the purpose are oxidized and reduced glutathione (GSH/GSSG), cysteine/cystine, cysteamine/cystamine, (DTT)/GSSG, and (DTE)/GSSG). In some other embodiments, the methods disclosed herein do not include a redox system such as, a thiol redox system. In some embodiments, one or more of the buffers (e.g., suspension buffer, the solubilization buffer, and folding buffer) in the methods disclosed herein does not contain a redox system. In some other embodiments, the folding buffer does not contain a redox system. In some embodiments, the folding buffer does not contain a glutathione redox system. In some embodiments, the folding buffer does not contain a cysteine/cystine redox system.

If desired, the protein concentration of a sample at any given step of the disclosed methods can be determined, and any suitable method can be employed. Such methods are well known in the art and include: 1) colorimetric methods such as the Lowry assay, the Bradford assay, the Smith assay, and the colloidal gold assay; 2) methods utilizing the UV absorption properties of proteins; and 3) visual estimation based on stained protein bands on gels relying on comparison with protein standards of known quantity on the same gel. Periodic determinations of protein concentration can be useful for monitoring the progress of the method as it is performed.

It is noted that any or all steps of the disclosed methods can be carried out manually or by any convenient automated means, such as by employing automated or computer-controlled systems.

As discussed above, an advantageous characteristic of the multi-step folding process disclosed herein is its scalability, which allows the methods of the disclosure to be practice on any scale, from bench scale or pilot scale to industrial or commercial scale. In particular, the disclosed methods will find suitable applications at the commercial scale, where it can be deployed to efficiently fold or refold large quantities of G-CSF.

### Compositions of the Disclosure

In one aspect, the present disclosure provides a granulocyte colony-stimulating factor (G-CSF) purified or isolated by methods disclosed herein. In some preferred embodiments, the purified or isolated G-CSF obtained by such methods is biologically active G-CSF.

The purified or isolated G-CSF obtained in accordance with the methods of the present disclosure, and particularly the biologically active G-CSF obtained by such methods, can be particularly suited for therapeutic applications. Accordingly, in one aspect of the disclosure, some embodiments disclosed herein relate to a pharmaceutical composition which includes a therapeutically effective amount of the biologically active G-CSF as disclosed herein and is suitable for therapeutic and clinical use.

The pharmaceutical compositions in accordance with the disclosure include compositions and formulations for human and veterinary use. In some embodiments, the pharmaceutical composition includes a mixture of the biologically active G-CSF as disclosed herein with a pharmaceutically acceptable auxiliary substance. Suitable pharmaceutically acceptable auxiliary substances include suitable diluents, adjuvants and/or carriers useful in G-CSF therapy. Non-limiting examples of pharmaceutically acceptable auxiliary substance include, but is not limited to, saline, solvents, dispersion media, coatings, antibacterial and antifungal agents, isotonic and absorption delaying agents, compatible with pharmaceutical administration. Supplementary active substances can also be incorporated into the compositions. In some embodiments, the pharmaceutical composition further includes pharmaceutically acceptable additives such as buffers, salts and stabilizers. The G-CSF and the pharmaceutical compositions obtained according to the present disclosure can either be (i) used directly or (ii) further processed, for instance pegylated as described in greater detail below or in, e.g., PCT Publication No. WO2008/124406 and then stored in the form of a powder or a lyophilisate or in liquid form. In some embodiments, the pharmaceutical composition of the disclosure is a liquid composition. In some embodiments, the pharmaceutical composition of the disclosure is a lyophilisate or a powder.

The G-CSF as an active ingredient of a pharmaceutical composition can be administered in a typical method through an intravenous, intra-arterial, intraperitoneal, intrastemal, transdermal, nasal, inhalant, topical, rectal, oral, intraocular or subcutaneous route. The administration method is not particularly limited, but a non-oral administration is preferable, and the subcutaneous or intravenously administration is more preferable.

In some embodiments, pharmaceutical compositions suitable for injectable use include sterile aqueous solutions (where water soluble) or dispersions and sterile powders for the extemporaneous preparation of sterile injectable solutions or dispersion. For intravenous administration, suitable auxiliary substance include physiological saline, bacteriostatic water, Cremophor ELTM (BASF, Parsippany, N.J.) or phosphate buffered saline (PBS). In some cases, the composition should be sterile and should be fluid to the extent that easy syringability exists. It should be stable under the conditions of manufacture and storage and must be preserved against the contaminating action of microorganisms such as bacteria and fungi. The auxiliary substance can be a solvent or dispersion medium containing, for example, water, ethanol, polyol (for example, glycerol, propylene glycol, and liquid polyethylene glycol), and suitable mixtures thereof. The proper fluidity can be maintained, for example, by the use of a coating such as lecithin, by the maintenance of the required particle size in the case of dispersion and by the use of surfactants, e.g., sodium dodecyl sulfate. Prevention of the action of microorganisms can be achieved by various antibacterial and antifungal agents, for example, parabens, chlorobutanol, phenol, ascorbic acid, thimerosal. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Additional examples of suitable adjuvants in the pharmaceutical compositions containing G-CSF as disclosed herein include, but are not limited to, stabilizers like sugar and sugar alcohols, amino acids and tensides like for example Polysorbate-20, Polysorbate-60, Polysorbate-65, Polysorbate-80, as well as suitable buffer substances. In some embodiments according to the methods of the present disclosure, the purified/isolated biologically active G-CSF is formulated in 10 mM acetic acid at a pH of 4.0, 0.0025% Polysorbate 80 and 50 g/L Sorbitol.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions, if used, generally include an inert diluent or an edible carrier. For the purpose of oral therapeutic administration, the active compound (e.g., G-CSF disclosed herein and/or pharmaceutical compositions containing the same) can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, and troches can contain any of the following ingredients, or compounds of a similar nature: a binder such as microcrystalline cellulose, gum tragacanth or gelatin; an excipient such as starch or lactose, a disintegrating agent such as alginic acid, Primogel™, or corn starch; a lubricant such as magnesium stearate or Sterotes™; a glidant such as colloidal silicon dioxide; a sweetening agent such as sucrose or saccharin; or a flavoring agent such as peppermint, methyl salicylate, or orange flavoring.

In the event of administration by inhalation, the subject G-CSF and/or pharmaceutical compositions as disclosed herein of the disclosure are delivered in the form of an aerosol spray from pressured container or dispenser which contains a suitable propellant, e.g., a gas such as carbon dioxide, or a nebulizer. Such methods include those described in, for example, U.S. Pat. No. 6,468,798.

Systemic administration of the subject G-CSF and/or pharmaceutical compositions as disclosed herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In some embodiments, the subject G-CSF and/or pharmaceutical compositions as disclosed herein can also be prepared in the form of suppositories (e.g., with conventional suppository bases such as cocoa butter and other glycerides) or retention enemas for rectal delivery. In some embodiments, the G-CSF and/or pharmaceutical compositions of the disclosure can also be administered by transfection or infection using methods known in the art.

In one embodiment, the pharmaceutical compositions of the disclosure are prepared with carriers that will protect the recombinant G-CSF against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art such as, for example, those described in U.S. Pat. No. 4,522,811.

In some embodiments, the recombinant G-CSF of the disclosure can be further modified to prolong their half-life in vivo and/or ex vivo. Non-limiting examples of known strategies and methodologies suitable for modifying the recombinant G-CSF of the disclosure include (1) chemical modification of a polypeptide described herein with highly soluble macromolecules such as polyethylene glycol (“PEG”) which prevents the polypeptides from contacting with proteases; and (2) covalently linking or conjugating a polypeptide described herein with a stable protein such as, for example, albumin. Accordingly, in some embodiments, the recombinant G-CSF of the disclosure can be fused to a stable protein, such as, albumin. For example, human albumin is known as one of the most effective proteins for enhancing the stability of polypeptides fused thereto and there are many such fusion proteins reported.

In some embodiments, the recombinant G-CSF of the disclosure are chemically modified with one or more polyethylene glycol moieties, e.g., PEGylated; or with similar modifications, e.g. PASylated. In some embodiments, the PEG molecule or PAS molecule is conjugated to one or more amino acid side chains of the interferon. In some embodiments, the PEGylated or PASylated G-CSF polypeptide contains a PEG or PAS moiety on only one amino acid. In other embodiments, the PEGylated or PASylated G-CSF polypeptide contains a PEG or PAS moiety on two or more amino acids, e.g., attached to two or more, five or more, ten or more, fifteen or more, or twenty or more different amino acid residues. In some embodiments, the PEG or PAS chain is 2000, greater than 2000, 5000, greater than 5,000, 10,000, greater than 10,000, greater than 10,000, 20,000, greater than 20,000, and 30,000 Da. The PASylated G-CSF polypeptide may be coupled directly to PEG or PAS (e.g., without a linking group) through an amino group, a sulfhydryl group, a hydroxyl group, or a carboxyl group. In some embodiments, the recombinant G-CSF of the disclosure is covalently bound to a polyethylene glycol with an average molecular weight of 20,000 Daltons.

In some embodiments, the pharmaceutical compositions of the disclosure include one or more pegylation reagent. As used herein, the term “PEGylation” refers to modifying a protein by covalently attaching polyethylene glycol (PEG) to the protein, with “PEGylated” referring to a protein having a PEG attached. A range of PEG, or PEG derivative sizes with optional ranges of from about 10,000 Daltons to about 40,000 Daltons may be attached to the recombinant polypeptides of the disclosure using a variety of chemistries. In some embodiments, the pegylation reagent is selected from methoxy polyethylene glycol-succinimidyl propionate (mPEG-SPA), mPEG-succinimidyl butyrate (mPEG-SBA), mPEG-succinimidyl succinate (mPEG-SS), mPEG-succinimidyl carbonate (mPEG-SC), mPEG-Succinimidyl Glutarate (mPEG-SG), mPEG-N-hydroxyl-succinimide (mPEG-NHS), mPEG-tresylate and mPEG-aldehyde. In some embodiments, the pegylation reagent is polyethylene glycol; preferably said pegylation reagent is polyethylene glycol with an average molecular weight of 20,000 Daltons covalently bound to the N-terminal methionine residue of the protein.

### Methods of Treatment

The purified or isolated G-CSF obtained in accordance with the methods of the present disclosure, and particularly the biologically active G-CSF obtained by such methods, can be particularly suited for therapeutic applications. In this context, some embodiments of the disclosure relate to a method for treating or preventing a disease in a subject including administering to the subject a therapeutically effective amount of a G-CSF as disclosed herein and/or a pharmaceutical composition as disclosed herein.

The terms “administration” and “administering”, as used herein, refer to the delivery of a bioactive composition or formulation by an administration route including, but not limited to, oral, intravenous, intra-arterial, intramuscular, intraperitoneal, subcutaneous, intramuscular, and topical administration, or combinations thereof. The term includes, but is not limited to, administering by a medical professional and self-administering. The term “therapeutically effective amount” used herein refers to the amount of biologically active G-CSF obtained by the methods disclosed herein which has the therapeutic effect of biologically active G-CSF.

In some embodiments of the disclosure, the G-CSF and/or pharmaceutical composition as disclosed herein is formulated to be compatible with its intended route of administration. The G-CSF and/or pharmaceutical composition of the disclosure may be given orally or by inhalation, but it is more likely that they will be administered through a parenteral route. Examples of parenteral routes of administration include, for example, intravenous, intradermal, subcutaneous, transdermal (topical), transmucosal, and rectal administration. Solutions or suspensions used for parenteral application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as EDTA; buffers such as acetates, citrates or phosphates and agents for the adjustment of tonicity such as sodium chloride or dextrose. pH can be adjusted with acids or bases, such as mono- and/or di-basic sodium phosphate, hydrochloric acid or sodium hydroxide (e.g., to a pH of about 7.2-7.8, e.g., 7.5). The parenteral preparation can be enclosed in ampoules, disposable syringes or multiple dose vials made of glass or plastic.

Dosage, toxicity and therapeutic efficacy of such subject G-CSF and/or pharmaceutical compositions of the disclosure can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in mammals, e.g., humans. The dosage of such compounds lies preferably within a range of circulating concentrations that include the ED₅₀ with little or no toxicity. The dosage may vary within this range depending upon the dosage form employed and the route of administration utilized. For any pharmaceutical compositions used in the treatment methods of the disclosure, the therapeutically effective dose can be estimated initially from cell culture assays. A dose may be formulated in animal models to achieve a circulating plasma concentration range that includes the IC₅₀ (e.g., the concentration of the test compound which achieves a half-maximal inhibition of symptoms) as determined in cell culture. Such information can be used to more accurately determine useful doses in humans. Levels in plasma may be measured, for example, by high performance liquid chromatography.

In some embodiments, the methods of the disclosure are suitable for the treatment and/or prevention of a disease associated with one or more indications selected from the group consisting of neutropenia and neutropenia-related clinical sequelae, chronic neutropenia, neutropenic and non-neutropenic infections, reduction of hospitalization for febrile neutropenia after cytotoxic chemotherapy and for the reduction in the duration of neutropenia in patients undergoing myeloablative therapy followed by bone marrow transplantation considered to be at increased risk of prolonged severe neutropenia. In some embodiments, the methods of the disclosure are suitable for the treatment and/or prevention of a disease associated with the mobilization of peripheral blood progenitor cells (PBPC) and chronic inflammatory conditions.

In some embodiments, long term administration of the G-CSF disclosed herein and/or pharmaceutical compositions containing the same is indicated to increase neutrophil counts and to reduce the incidence and duration of infection-related events, treatment of persistent neutropenia in patients with advanced HIV infection, in order to reduce the risk of bacterial infections. In some embodiments, the G-CSF disclosed herein and/or pharmaceutical compositions containing the same is indicated for improving the clinical outcome in intensive care unit patients and critically ill patients, wound/skin ulcers/burns healing and treatment, intensification of chemotherapy and/or radiotherapy, increase of anti-inflammatory cytokines, potentiation of the antitumor effects of photodynamic therapy. In some embodiments, the G-CSF disclosed herein and/or pharmaceutical compositions containing the same is indicated for prevention and treatment of illness caused by different cerebral dysfunctions, treatment of thrombotic illness and their complications and post irradiation recovery of erythropoiesis. It can be also used for treatment of all other illnesses reported as indicative for G-CSF.

A pharmaceutical composition containing the biologically active G-CSF obtained by the methods disclosed herein can thus be administered, to patients, children or adults in a therapeutically amount which is effective to treat or prevent one or more of the above mentioned diseases. In some embodiments, the methods of the disclosure are suitable for the treatment and/or prevention of neutropenia.

All publications and patent applications mentioned in this disclosure are herein incorporated by reference to the same extent as if each individual publication or patent application was specifically and individually indicated to be incorporated by reference.

No admission is made that any reference cited herein constitutes prior art. The discussion of the references states what their authors assert, and the inventors reserve the right to challenge the accuracy and pertinence of the cited documents. It will be clearly understood that, although a number of information sources, including scientific journal articles, patent documents, and textbooks, are referred to herein; this reference does not constitute an admission that any of these documents forms part of the common general knowledge in the art.

The discussion of the general methods given herein is intended for illustrative purposes only. Other alternative methods and alternatives will be apparent to those of skill in the art upon review of this disclosure, and are to be included within the spirit and purview of this application.

### EXAMPLES

In the Examples described below, a number of G-CSF folding conditions were evaluated and optimized. The G-CSF described in these Examples was a recombinant human granulocyte colony-stimulating factor (rhG-CSF) with two intra-molecular disulfide bonds. The sequence of the recombinant rhG-CSF is identical to the natural human granulocyte colony-stimulating factor sequence except for the inclusion of an N-terminal methionine due to expression in Escherichia coli. In these experiments, rhG-CSF is a non-glycosylated protein.

All scale-down purification experiments described below were performed using AKTA Explorers (GE Healthcare) and Omnifit chromatography columns. An Agilent UV-Vis or Thermo Nanodrop was used to determine in-process chromatography pool concentrations. Purification of the quarter scale and the 100 L experimental and engineering material was performed with an AKTA Ready and AKTA Pilot using BPG chromatography columns. Ultrafiltration/diafiltration (UF/DF) was performed with a manual system for the quarter scale and an automated Millipore Mobius skid for the experimental and engineering runs.

### Example 1

### Development of Solubilization and Folding Process

This Example illustrates a non-limiting exemplary workflow in which rhG-CSF contained in inclusion bodies was solubilized and then folded to yield biologically active rhG-CSF protein. The main principle of the newly described methods involves a multi-step folding process developed for rhG-CSF which includes: (i) dispersing of the IBs containing G-CSF in a suspension buffer, (ii) solubilization of the rhG-CSF contained in the IBs by using a denaturing agent (sarkosyl), (iii) use of folding buffer containing only the reduced form of a thiol redox pair to initiate folding of the solubilized rhG-CSF, and (iv) reduction of the denaturing agent concentration by a series of dilution steps. As discussed above, the multi-step folding process disclosed herein can be advantageously implemented at a commercial production scale.

In a typical experimental workflow, the frozen inclusion body pellet was broken up during the suspension stage with a blender in a Tris suspension buffer (40 mM Tris, pH 7.6) at 25 gram of buffer per gram of pellet mass. The frozen pellets were blended with the suspension buffer (25 mL/g of frozen pellet) for 20 seconds, followed by gentle mixing for 15 minutes at room temperature. This step expedited the thawing of the inclusion bodies prior to the solubilization step. After blending, the inclusion bodies suspension was transferred to a closed disposable container with agitator mixing.

The subsequent solubilization step was then performed with sarkosyl used as the denaturing agent. The inclusion bodies were dissolved with a solubilization buffer (40 mM Tris, Sarkosyl, pH 8.4) which was added at a volume ratio of 1:1 with the suspension buffer. In some embodiments, the solubilization buffer was added at 25 mL/g of lysate pellet. Without being bound to any particular theory, the denaturing agent is believed to unfold the inclusion bodies and reduce susceptibility to aggregation. In these experiments, sarkosyl concentrations of 0.56%, 1.0%, and 2.0% were evaluated for solubilization. It was observed that approximately 5 g of inclusion bodies were completely solubilized in 2 hours with the addition of a solubilization buffer containing 0.56%, 1.0%, or 2.0% sarkosyl.

After incubation for 14 to 24 hours, the solubilization buffer in the solubilizate was further diluted at a volume ratio of 1:1 with WFI, and a folding buffer (40 mM Tris, 0.8 mM Cysteine, pH 7.8) is added to a final cysteine concentration of 80 μM to initiate folding. Generally, 10 mL of folding buffer was added for one gram of lysate pellet. The folding mixture was then mixed for about 15 minutes at room temperature, followed by incubation without mixing at 15-25° C. for 22±2 hours. The folding mixture was then diluted 1:1 with WFI and further mixed about 15 minutes.

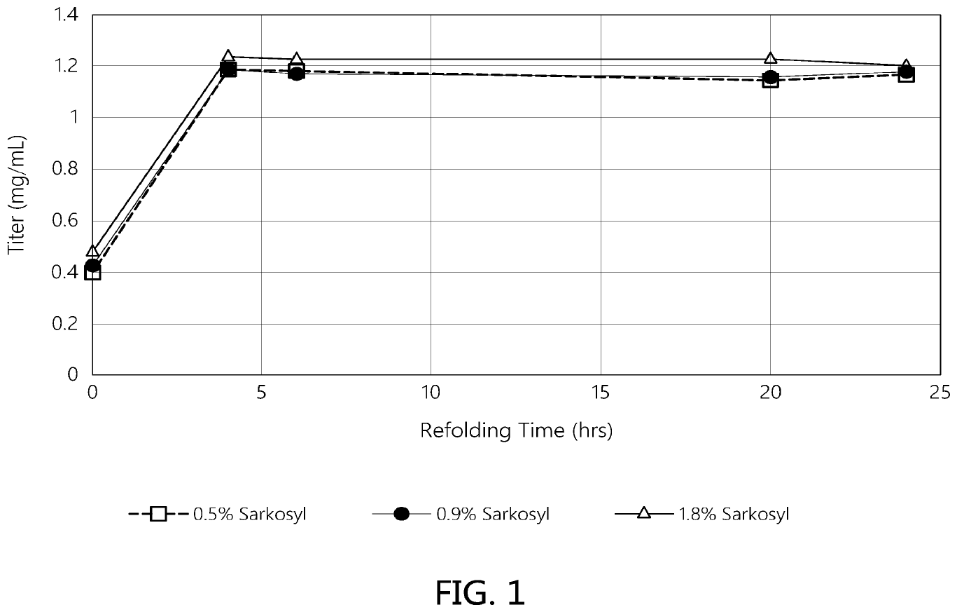

This addition of folding buffer decreased the sarkosyl levels for folding by dilution to 0.5%, 0.9%, and 1.8% respectively. Samples were analyzed for titer by reversed phase HPLC at 0, 4, 6, 20, and 24 hours. Data shown in FIG. 1 indicated that solubilization and folding operation could be performed at all three of these buffer sarkosyl concentrations resulting in a similar quantity of protein and similar refolding rates.

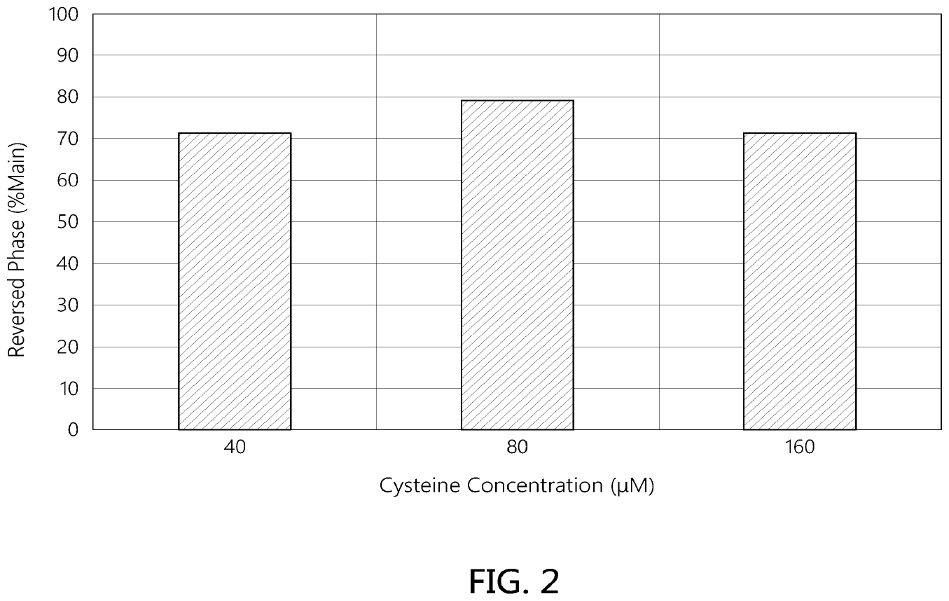

It was further observed that cysteine was effective in initiating folding at three concentrations of 40 μM, 80 μM, and 160 μM at a 0.5% sarkosyl concentration as assayed by reversed phase HPLC (RP-HPLC; see FIG. 2).

The removal of sarkosyl from the folding mixture was achieved using Dowex 1×8 ion exchange resin (50-100 mesh; 54 g/1 kg folding mixture) with a wash buffer containing 40 mM Tris, pH 7.7, for 3 hours at room temperature. The Dowex 1×8 ion exchange resin was subsequently removed by straining. In a further optional recovery step, washed G-CSF protein was passed through a depth filter.

### Example 2

### Optimization of Sequential Stepwise Reduction of Sarkosyl Content

This Example describes experiments performed to optimize the sequential stepwise reduction of sarkosyl concentration during preparation of the rhG-CSF in accordance with some embodiments of the disclosure. In these experiments, several parameters were evaluated in the sarkosyl folding experiments through three sets of refinement experiments. These parameters include (i) the gram ratio of sarkosyl to protein, (ii) the use of EDTA, glutathione or cysteine at solubilization step, (iii) the use of cysteine, glutathione, or a redox system at folding step, (iv) percent of sarkosyl at folding step, (v) percent sarkosyl at resin removal step, and (vi) quantity of Dowex.

Parameters that were not altered in these experiments include folding at room temperature, the use of 1.0% sarkosyl buffer for solubilization, solubilization time, folding time, and detergent removal resin and mix time.

For the initial folding studies, washed USU Run 11 inclusion body pellet was used. Each condition was evaluated with 20 mg of D3. After folding, samples were analyzed RP-HPLC titer. Several iterations of studies occurred with Run 11 D3 to evaluate combinations of conditions and to ensure consistency (data not shown). A range of conditions were then evaluated with Run 9 IB to confirm consistency across feedstocks. Data from the Run 9 set of experiments is shown in TABLE 1.

TABLE 1

| | | | | | | |
|---|---|---|---|---|---|---|
| Condition | 1 | 2 | 3 | 4 | 5 | 6 |

Solubilization (Mix time 3 hr)

| Sarkosyl | 1% | 1% | 1% | 1%* | 1% | | 1% | |
|---|---|---|---|---|---|---|---|---|
| Sarkosyl (g):Protein (g) | 10:1 | 10:1 | 10:1 | 5:1 | 5:1 | | 5:1 | |
| Additives | 0 | EDTA 1 mM | 0 | 0 | 100 | mM GSH | 5 | mM Cys |

Folding (24-hr no Mixing)

| Sarkosyl | 1% | 1% | 0.5% | 0.5% | | 0.5% | 0.5% |
|---|---|---|---|---|---|---|---|
| Cysteine | 80 μM | 80 μM | 0 | 80 | μM | 0 | 0 |

Post-Folding

| Dilution | 0 | 0 | 0 | 2x | | 2x | | 2x | |
|---|---|---|---|---|---|---|---|---|---|
| Hold time | N/A | N/A | N/A | 15 | min | 15 | min | 15 | min |

Detergent Removal (Mix time 3 hr)

| | | | | | | |
|---|---|---|---|---|---|---|
| Dowex (mg) | 54 | 108 | 51 | 108 | 54 | 54 |

Non-titrated, non-reduced analytics

| | | | | | | |
|---|---|---|---|---|---|---|
| Amount (all peaks) | 1.43 | 1.25 | 0.69 | 0.68 | 0.69 | 0.57 |
| mg/mL | | | | | | |
| Amount (non-reduced) | 1.00 | 1.04 | 0.30 | 0.54 | 0.59 | 0.18 |
| mg/mL | | | | | | |
| Amount (reduced) | 0.03 | 0.03 | 0.32 | 0.02 | 0.02 | 0.03 |
| mg/mL | | | | | | |
| Rel. Area Non-Reduced | 70.06 | 83.39 | 43.39 | 78.69 | 84.23 | 30.98 |
| % | | | | | | |
| Multiplier | 1 | 1 | 1 | 2 | 2 | 2 |
| Amount (all peaks) | 1.43 | 1.25 | 0.69 | 1.36 | 1.38 | 1.14 |
| mg/mL | | | | | | |
| Amount (non-reduced) | 1.00 | 1.04 | 0.30 | 1.08 | 1.18 | 0.36 |
| mg/mL | | | | | | |

Titrated, non-reduced analytics

| | | | | | | |
|---|---|---|---|---|---|---|
| Amount (all peaks) | 0.91 | 1.19 | 0.14 | 0.62 | 0.66 | 0.51 |
| mg/mL | | | | | | |
| Amount (non-reduced) | 0.68 | 1.03 | 0.08 | 0.53 | 0.57 | 0.17 |
| mg/mL | | | | | | |
| Amount (reduced) | 0.01 | 0.01 | 0.05 | 0.01 | 0.01 | 0.01 |
| mg/mL | | | | | | |
| Rel. Area Non-Reduced | 75.21 | 86.53 | 60.65 | 85.85 | 86.80 | 33.58 |
| % | | | | | | |
| Multiplier | 1 | 1 | 1 | 2 | 2 | 2 |
| Amount (all peaks) | 0.91 | 1.19 | 0.14 | 1.24 | 1.32 | 1.02 |
| mg/mL | | | | | | |
| Amount (non-reduced) | 0.68 | 1.03 | 0.08 | 1.06 | 1.14 | 0.34 |
| mg/mL | | | | | | |

*suspended in Tris buffer prior to addition of sarkosyl.

The data was evaluated by comparing the overall quantity of protein and the percent non-reduced. In the data set presented in TABLE 1, the first condition is the control using the parameters of the current at-scale runs at that time. It was observed that Condition 4 and Condition 5 yielded the most promising results. In particular, only the reduced form glutathione (L-GSH) was used in Condition 5 (e.g., the oxidized form of glutathione—GSSG—was excluded from this reaction). On the other hand, Condition 4 did not require the addition of any new raw materials into the production (unlike Condition 5). Impacts to the absence or inclusion of certain additives could be observed in the chromatographic data. When cysteine was present at solubilization step, two peaks of lower hydrophobicity can be observed. When no cysteine was added in the folding mixture, reduced G-CSF remained (data not shown). A small scale confirmation was performed with the best folding condition (see, e.g., Example 3 below).

### Example 3

### Small Scale Confirmation of Folding

The best folding conditions identified in a screening format as described in Example 2 above were further evaluated in small scale processing. The control with suspension is a slight modification of the control condition described in Example 2 where the inclusion bodies were suspended in Tris buffer first and the Dowex is increased to 10 g per gram of sarkosyl, but all other handling and ratios remained the same as in prior processing. The Tris suspension condition corresponded to Condition 4 in Example 2. The study design is described in TABLE 2.

TABLE 2

| | Control with | Tris | Tris |
|---|---|---|---|
| | Suspension | Suspension | Suspension |
| USU Run | 6 | 6 | 9 |
| Suspension | Tris buffer | Tris buffer | Tris buffer |
| Solubilization | 1% Sarkosyl | 1% Sarkosyl | 1% Sarkosyl |
| | 10:1 ratio | 5:1 ratio | 5:1 ratio |
| Folding | 1% Sarkosyl | 0.5% Sarkosyl | 0.5% Sarkosyl |
| | 80 μM Cys | 80 μM Cys | 80 μM Cys |
| Post-folding | N/A | 2x WFI | 2x WFI |
| Dowex | 10 g/g Sarkosyl | 10 g/g Sarkosyl | 10 g/g Sarkosyl |
| Chromatography | DEAE | DEAE | DEAE |

Final protein yields from the small scale processing study are provided in TABLE 3 below.

TABLE 3

| | Control with | Tris | Tris |
|---|---|---|---|
| | Suspension | Suspension | Suspension |
| G-CSF Mass Used (mg) | 923.9 | 969.9 | 736.9 |
| Folded G-CSF Mass (mg) | 454.4 | 611.5 | 418.1 |
| DEAE Yield (%) | 22.5 | 37.0 | 43.3 |
| CM Yield (%) | 56.2 | 82.0 | N/T |
| Overall Yield (%) | 11.1 | 23.1 | 24.6 |

The Tris suspension condition demonstrated yield consistency between feedstocks and a 2-fold increase in yield over the control condition. This condition was selected to move forward with in a quarter scale production, experimental, and engineering runs. Due to modifications to the control, yield improvement is most likely not influenced by the initial suspension with Tris or the quantity of Dowex for sarkosyl removal. Without being bound to any particular theory, important parameters are believed to include (1) the ratio of sarkosyl to protein, (2) the percent sarkosyl at folding, and (3) the dilution of the folding mixture prior to sarkosyl removal.

### Example 4

### Kinetics of Folding

This Example describes experiments performed to evaluate the effects of folding time on the G-CSF product quality. In these experiments, folding time was evaluated using samples from two at-scale engineering lots.

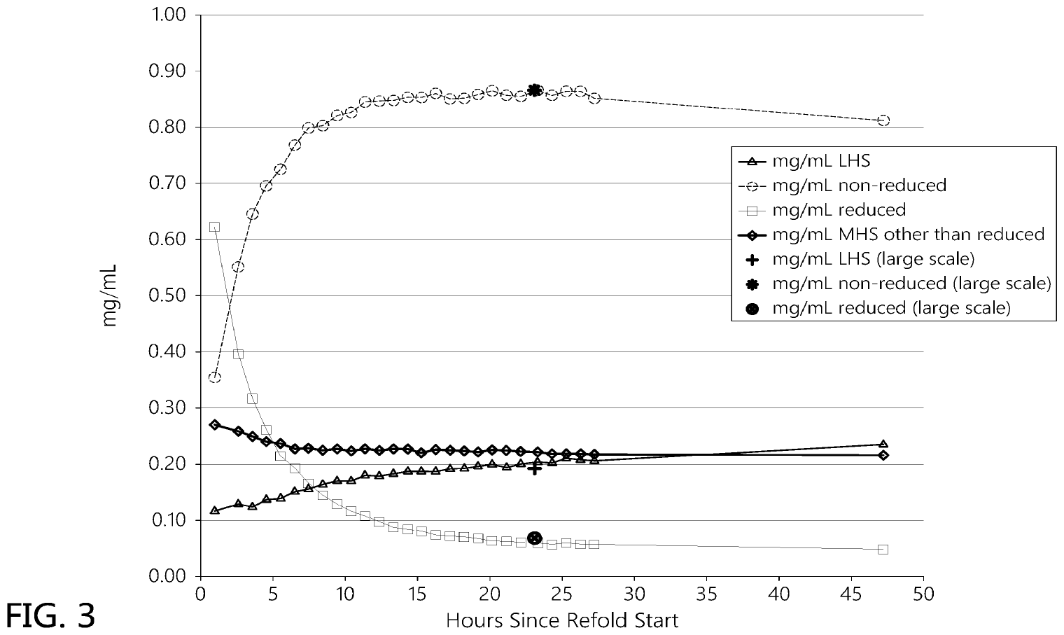

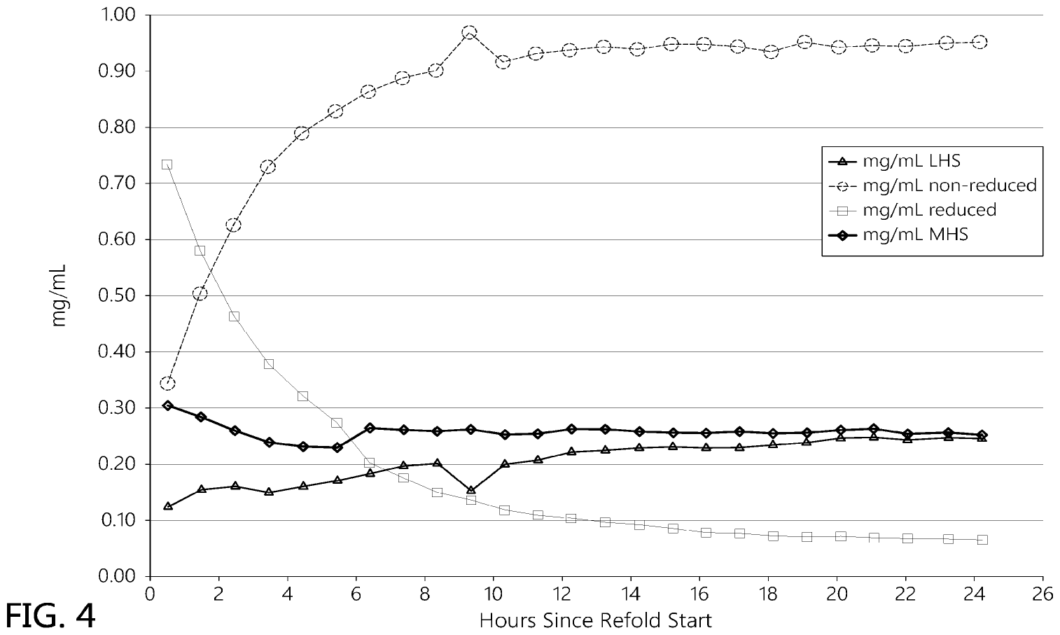

In these experiments, the folding rate was evaluated with a sample from two engineering runs: Engineering 1 and Engineering 2. Folding for these two engineering runs was according to parameters described in Example 3 above. The samples were pulled from the bulk at the start of folding and placed in an auto-sampler at 22° C. A reversed phase injection was performed at selected time points, and the data is presented in FIG. 3 and FIG. 4. The reversed phase data for the endpoint of the bulk Engineering 1 at 22 hours is represented on FIG. 3. The data was similar for both datasets indicating the auto-sampler time-course approach is representative. For both datasets, the folding process was complete within 12 to 14 hours. Due to the completion earlier than the 22 hour duration, there was an interest in Engineering 2 to perform a small scale experiment to determine if there was a benefit to reducing the folding time. A sample was pulled from Engineering 2 at the folding stage and carried through the process to CM fractionation after a 15 hour folding time. Data obtained from this experiment was comparable to the longer folding time at the larger scale. Resulting data from 15 hour folding is shown in TABLE 4.

TABLE 4
Kinetics of folding.

| | | Reversed Phase | | | IEC | | | SEC | | |
|---|---|---|---|---|---|---|---|---|---|---|
| Operation | % Yield | % LHS | % Main | % MHS | % Acidic | % Main | % Basic | % HMW | % Main | % LMW |
| DEAE | 65 | 2.35 | 93.73 | 3.92 | 4.45 | 95.55 | 0.00 | 0.5 | 99.5 | 0.0 |
| CM | 111 | 1.98 | 96.43 | 1.59 | 2.01 | 97.99 | 0.00 | 0.2 | 98.8 | 0.0 |
| Neupogen | N/A | 2.40 | 93.64 | 3.96 | 1.98 | 98.02 | 0.00 | N/T | N/T | N/T |

LHS: Less hydrophobic species. MHS: More hydrophobic species. HMW: High molecular weight species. LMW: Low molecular weight species. IEC: Ion-exchange chromatography. SEC: Size-exclusion chromatography.

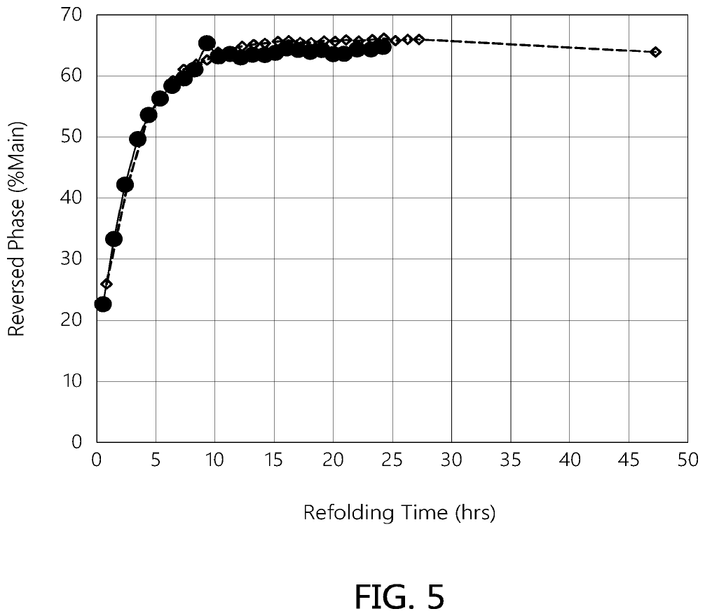

FIG. 5 is an overlay of the data sets for the reversed phase HPLC % main from FIG. 3 and FIG. 4, which demonstrates the consistency of the methods disclosed herein. A consistent rate in folding as indicated in the change in % main by reversed phase HPLC is observed in Engineering 1 and Engineering 2. The folding process for both source materials was complete within 12 to 14 hours.

### Example 5

### Temperature of Folding

In these experiments, a small study was conducted to evaluate folding at 4° C. A sample was pulled from Experiment 2 at the start of folding at ambient temperature. This sample was placed at 4° C. for the same folding duration. Folding efficiency was determined by reversed phase analysis. Data in TABLE 5 indicates there was a slight improvement in folding at the chilled temperature, but this may be in range of variability.

TABLE 5

| | All peaks | Non-reduced | Reduced | Non-reduced | Reduced |
|---|---|---|---|---|---|
| | (mg/mL) | (mg/mL) | (mg/mL) | (%) | (%) |
| Refold | 0.50 | 0.38 | 0.03 | 75.88 | 5.73 |
| Room | | | | | |
| Temp | | | | | |
| Refold | 0.43 | 0.36 | 0.02 | 83.78 | 4.22 |
| 4° C. | | | | | |

### Example 6

### Cysteine Stability

In all productions of G-CSF, the folding buffer 40 mM Tris, 0.8 mM Cysteine, pH 7.8 was prepared the day of folding initiation. This would potentially be challenging in manufacturing, so a cysteine stability study was performed. A sample of cysteine buffer was taken from engineering run 1. This buffer was stored at room temperature. At certain time-points, the stored buffer was used to initiate a folding of a USU run 9 D3 pellet. Percent non-reduced was then measured by reverse phase titer. This was used to determine the stability of the buffer. The study indicated that the cysteine buffer could be stored at room temperature for at least two weeks.

While particular alternatives of the present disclosure have been disclosed, it is to be understood that various modifications and combinations are possible and are contemplated within the true spirit and scope of the appended claims. There is no intention, therefore, of limitations to the exact abstract and disclosure herein presented.

What is claimed is:

1. A method for isolating and/or purifying granulocyte colony-stimulating factor (G-CSF) from inclusion bodies (IBs), comprising:
a) solubilizing the G-CSF contained in the IBs with a solubilization buffer comprising a denaturing agent; and
b) initiating folding of the solubilized G-CSF by diluting, via a sequential stepwise dilution process, the solubilizate from (a) with a folding buffer comprising only the reduced form of a thiol redox pair to obtain a folding mixture comprising folded G-CSF.

2. A method for preparing biologically active granulocyte colony-stimulating factor (G-CSF), comprising:
a) solubilizing inclusion bodies (IBs) containing G-CSF with a solubilization buffer comprising a denaturing agent; and
b) initiating folding of the solubilized G-CSF by diluting, via a sequential stepwise dilution process, the solubilizate from (a) with a folding buffer comprising only the reduced form of a thiol redox pair to obtain a folding mixture comprising folded G-CSF with improved purity and/or functional activity.

3. The method of claim 2, wherein the obtained G-CSF comprises biologically active, correctly folded G-CSF with a purity of greater than 80%.

4. The method of any one of claims 1 to 2, further comprising recovering the folded G-CSF.

5. The method of any one of claims 1 to 4, wherein the IBs containing G-CSF are suspended in a suspension buffer prior to solubilization.

6. The method of claim 5, wherein the suspension buffer comprises about 20 mM to 60 mM Tris at pH ranging from about 7.0 to 8.0.

7. The method of claim 6, wherein the suspension buffer comprises about 40 mM Tris at pH of about 7.6.

8. The method of any one of claims 1 to 7, wherein the denaturing agent in the solubilization buffer comprises a mild denaturing detergent, a strong denaturing detergent, an ionic detergent, or any combination thereof.

9. The method of any one of claims 1 to 8, wherein the denaturing agent in the solubilization buffer comprises N-lauroyl sarcosine (sarkosyl), sodium dodecyl sulfate (SDS), sodium lauryl sulfate, polyoxyethylene poloxypropylene glycol, cocoamphoacetate, lithium dodecyl sulfate, sodium octyl sulfate, deoxycholic acid, sodium cholate hydrate, sodium deoxycholate, sodium glycocholate, sodium taurodeoxycholate, or any combination thereof.

10. The method of any one of claims 1 to 9, wherein the denaturing agent comprises an anionic detergent.

11. The method of claim 10, wherein the anionic detergent in the solubilizing buffer is sarkosyl.

12. The method of claim 11, wherein sarkosyl is present in the solubilization buffer in an amount ranging from about 0.2% to about 5.0% by weight.

13. The method of any one of claims 11 to 12, wherein sarkosyl is present in the solubilization buffer in an amount of about 0.2%, about 0.56%, about 1.0%, or about 2.0% by weight.

14. The method of any one of claims 1 to 13, wherein the solubilization buffer comprises about 20 mM to 60 mM Tris, about 0.2% to about 5.0% sarkosyl, at pH ranging from about 7.5 to about 9.0.

15. The method of claim 14, wherein the solubilization buffer comprises about 40 mM, about 2.0% sarkosyl, at pH of about 8.4.

16. The method of any one of claims 5 to 15, wherein the volume ratio of the suspension buffer to the solubilization buffer is adjusted such that the final pH is about 7.5 to about 7.8.

17. The method of any one of claims 1 to 16, wherein the sequential stepwise dilution process comprises gradually reducing the concentration of the denaturing agent in the solubilizate from (a).

18. The method of claim 17, wherein the process of gradually reducing the denaturing agent concentration comprises one or more of the following:
(i) mixing the solubilization buffer with the suspension buffer in which the IBs containing G-CSF are suspended;
(ii) diluting the solubilizate from (i) with water for injection (WFI) to form a diluted solubilizate;
(iii) adding the folding buffer to the diluted solubilizate from (ii) to obtain a folding mixture; and
(iv) further diluting the folding mixture from (iii) with WFI.

19. The method of claim 18, wherein the volume ratio of the solubilization buffer to the suspension buffer in (i) is about 1:1.

20. The method of any one of claims 18 to 19, wherein the volume ratio of the solubilizate from (i) to WFI is about 1:1.

21. The method of any one of claims 18 to 20, wherein the volume ratio of the folding buffer to the diluted solubilizate from (ii) is about 1:1.

22. The method of any one of claims 18 to 21, wherein the volume ratio of the folding mixture from (iii) to WFI is about 1:1.

23. The method of any one of claims 1 to 22, wherein the reduced form of a thiol redox pair is the reduced form of cysteine, glutathione, penicillamine, N-acetyl-penicillamine, 2-mercaptoacetic acid, 2-mercaptopropionic acid, 3-mercaptopropionic acid, mercaptosuccinic acid, mercaptopyruvate, mercaptoethoanol, monothioglycerol, γ-glutamylcysteine, cysteinylglycine, cysteamine, N-acetyl-L-cysteine, homocysteine, or lipoic acid (dihydrolipoamide).

24. The method of any one of claims 1 to 23, wherein the reduced form of a thiol redox pair is reduced glutathione (GSH).

25. The method of any one of claims 1 to 23, wherein the reduced form of a thiol redox pair is cysteine.

26. The method of claim 24, wherein the cysteine is present in the folding buffer at a concentration ranging from about 20 μM to 200 μM.

27. The method of any one of claims 25 to 26, wherein the cysteine is present in the folding buffer at a concentration of about 40 μM, about 50 μM, about 80 μM, or about 160 μM.

28. The method of any one of claims 25 to 27, wherein the folding buffer is added to the solubilizate to a final concentration of cysteine of about 80 μM in the folding mixture.

29. The method of any one of claims 4 to 28, wherein the recovery of the folded G-CSF comprises one or more techniques selected from the group consisting of affinity chromatography, anion exchange chromatography (AEX), cation exchange chromatography (CEX), hydroxyapatite chromatography, size exclusion chromatography (SEC), hydrophobic interaction chromatography (HIC), metal affinity chromatography, mixed mode chromatography (MMC), centrifugation, diafiltration, and ultrafiltration.

30. The method of claim 29, wherein the anion exchange chromatography comprises DEAE Sepharose chromatography.

31. The method of claim 29, wherein the cation exchange chromatography comprises CM Sepharose chromatography.

32. The method of claim 29, wherein the diafiltration and/or ultrafiltration comprises a polyether sulfone membrane.

33. The method of any one of claims 1 to 32, wherein the G-CSF is a human G-CSF (hG-CSF).

34. The method of any one of claims 1 to 33, wherein the IBs are obtained from a recombinant cell expressing G-CSF wherein the expressed G-CSF forms the IBs in the cell.

35. The method of claim 34, wherein the recombinant cell is a prokaryotic cell or a eukaryotic cell.

36. The method of any one of claims 1 to 35, wherein the method does not comprise a strong denaturing agent, a strong reducing agent, a redox reaction, and/or a heavy metal.

37. The method of claim 36, wherein the strong reducing agent is urea or dithiothreitol (DTT).

38. The method of claim 36, wherein the heavy metal is copper.

39. A granulocyte colony-stimulating factor (G-CSF) purified or isolated by a method according to any one of claims 1 to 38.

40. A pharmaceutical composition comprising a therapeutically effective amount of the G-CSF of claim 39, and a pharmaceutically acceptable auxiliary substance.

41. The pharmaceutical composition of claim 40, wherein the pharmaceutical compositions is a liquid composition, a lyophilisate, or a powder.

42. A method for treating or preventing a disease in a subject comprising administering to the subject a therapeutically effective amount of the G-CSF of claim 39 and/or a pharmaceutical composition of claims 40-41.

43. The method of claim 42, wherein the disease is neutropenia.
